SEQUENCE LISTING (20 sequences)

SEQ ID NO: 1 - protein, 685 aa, Artificial Sequence
  Mos1.Env mosaic antigen sequence
MRVTGIRKNYQHLWRWGTMLLGILMICSAAGKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDDVRNVTNNATNTNSSWGEPMEKGEIKNCSFNITTSIRNKVQKQYALFYKLDVVPIDNDSNNTNYRLISCNTSVITQACPKVSFEPIPIHYCAPAGFAILKCNDKKFNGTGPCTNVSTVQCTHGIRPVVSTQLLLNGSLAEEEVVIRSENFTNNAKTIMVQLNVSVEINCTRPNNNTRKSIHIGPGRAFYTAGDIIGDIRQAHCNISRANWNNTLRQIVEKLGKQFGNNKTIVFNHSSGGDPEIVMHSFNCGGEFFYCNSTKLFNSTWTWNNSTWNNTKRSNDTEEHITLPCRIKQIINMWQEVGKAMYAPPIRGQIRCSSNITGLLLTRDGGNDTSGTEIFRPGGGDMRDNWRSELYKYKVVKIEPLGVAPTKAKRRVVQSEKSAVGIGAVFLGFLGAAGSTMGAASMTLTVQARLLLSGIVQQQNNLLRAIEAQQHLLQLTVWGIKQLQARVLAVERYLKDQQLLGIWGCSGKLICTTTVPWNASWSNKSLDKIWNNMTWMEWEREINNYTSLIYTLIEESQNQQEKNEQELLELDKWASLWNWFDISNWLW

SEQ ID NO: 2 - protein, 711 aa, Artificial Sequence
  Mos2SEnv mosaic antigen sequence
MRVRGMLRNWQQWWIWSSLGFWMLMIYSVMGNLWVTVYYGVPVWKDAKTTLFCASDAKAYEKEVHNVWATHACVPTDPNPQEIVLGNVTENFNMWKNDMVDQMHEDIISLWDASLEPCVKLTPLCVTLNCRNVRNVSSNGTYNIIHNETYKEMKNCSFNATTVVEDRKQKVHALFYRLDIVPLDENNSSEKSSENSSEYYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNETVNITCTRPNNNTRKSIRIGPGQTFYATGDIIGDIRQAHCNLSRDGWNKTLQGVKKKLAEHFPNKTIKFAPHSGGDLEITTHTFNCRGEFFYCNTSNLFNESNIERNDSIITLPCRIKQIINMWQEVGRAIYAPPIAGNITCRSNITGLLLTRDGGSNNGVPNDTETFRPGGGDMRNNWRSELYKYKVVEVKPLGVAPTEAKRRVVEREKRAVGIGAVFLGILGAAGSTMGAASITLTVQARQLLSGIVQQQSNLLRAIEAQQHMLQLTVWGIKQLQTRVLAIERYLQDQQLLGLWGCSGKLICTTAVPWNTSWSNKSQTDIWDNMTWMQWDKEIGNYTGEIYRLLEESQNQQEKNEKDLLALDSWNNLWNWFSISKWLWYIKIFIMIVGGLIGLRIIFAVLSIVNRVRQGY

SEQ ID NO: 3 - protein, 1350 aa, Artificial Sequence
  Mos1.Gag-Pol mosaic antigen sequence
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQMAPISPIETVPVKLKPGMDGPRVKQWPLTEEKIKALTAICEEMEKEGKITKIGPENPYNTPVFAIKKKDSTKWRKLVDFRELNKRTQDFWEVQLGIPHPAGLKKKKSVTVLAVGDAYFSVPLDEGFRKYTAFTIPSTNNETPGIRYQYNVLPQGWKGSPAIFQCSMTRILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVAGAANRETKLGKAGYVTDRGRQKIVSLTETTNQKTALQAIYLALQDSGSEVNIVTASQYALGIIQAQPDKSESELVNQIIEQLIKKERVYLSWVPAHKGIGGNEQVDKLVSSGIRKVLFLDGIDKAQEEHEKYHSNWRAMASDFNLPPVVAKEIVASCDQCQLKGEAMHGQVDCSPGIWQLACTHLEGKIILVAVHVASGYIEAEVIPAETGQETAYFILKLAGRWPVKVIHTANGSNFTSAAVKAACWWAGIQQEFGIPYNPQSQGVVASMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIKDYGKQMAGADCVAGRQDED

SEQ ID NO: 4 - protein, 1341 aa, Artificial Sequence
  Mos2.Gag-Pol mosaic antigen sequence
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSHKARVLAEAMSQTNSTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLRSLFGSDPLSQMAPISPIETVPVKLKPGMDGPKVKQWPLTEEKIKALVEICTEMEKEGKISKIGPENPYNTPIFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWEAWWTEYWQATWIPEWEFVNTPPLVKLWYQLEKEPIVGAETFYVAGAANRETKLGKAGYVTDRGRQKVVSLTDTTNQKTALQAIHLALQDSGLEVNIVTASQYALGIIQAQPDKSESELVSQIIEQLIKKEKVYLAWVPAHKGIGGNEQVDKLVSRGIRKVLFLDGIDKAQEEHEKYHSNWRAMASEFNLPPIVAKEIVASCDKCQLKGEAIHGQVDCSPGIWQLACTHLEGKVILVAVHVASGYIEAEVIPAETGQETAYFLLKLAGRWPVKTIHTANGSNFTSATVKAACWWAGIKQEFGIPYNPQSQGVVASINKELKKIIGQVRDQAEHLKTAVQMAVFIHNFKRKGGIGEYSAGERIVDIIASDIQTKELQKQITKIQNFRVYYRDSRDPLWKGPAKLLWKGEGAVVIQDNSDIKVVPRRKAKIIRDYGKQMAGDDCVASRQDED

SEQ ID NO: 5 - DNA, 2055 bp, Artificial Sequence
  Mos1.Env mosaic antigen sequence
atgcgggtgaccggcatccggaagaactaccagcacctgtggcggtggggcaccatgctgctgggcatcctgatgatttgctctgccgccggaaagctgtgggtgaccgtgtactacggcgtgcccgtgtggaaagaggccaccaccaccctgttctgcgccagcgacgccaaggcctacgacaccgaggtgcacaacgtgtgggccacccacgcctgcgtgcccaccgaccccaacccccaggaagtggtcctggaaaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacgacgtgcggaacgtgaccaacaacgccaccaacaccaacagcagctggggcgagcctatggaaaagggcgagatcaagaactgcagcttcaacatcaccacctccatccggaacaaggtgcagaagcagtacgccctgttctacaagctggacgtggtgcccatcgacaacgacagcaacaacaccaactaccggctgatcagctgcaacaccagcgtgatcacccaggcctgccccaaggtgtccttcgagcccatccccatccactactgcgcccctgccggcttcgccatcctgaagtgcaacgacaagaagttcaacggcaccggcccctgcaccaacgtgagcaccgtgcagtgcacccacggcatccggcccgtggtgtccacccagctgctgctgaacggcagcctggccgaggaagaggtggtgatcagaagcgagaatttcaccaacaatgccaagaccatcatggtgcagctgaacgtgagcgtggagatcaactgcacccggcccaacaacaacacccggaagagcatccacatcggccctggcagggccttctacacagccggcgacatcatcggcgacatccggcaggcccactgcaacatcagccgggccaactggaacaacaccctgcggcagatcgtggagaagctgggcaagcagttcggcaacaacaagaccatcgtgttcaaccacagcagcggcggagaccccgagatcgtgatgcacagcttcaactgtggcggcgagttcttctactgcaacagcaccaagctgttcaacagcacctggacctggaacaactccacctggaataacaccaagcggagcaacgacaccgaagagcacatcaccctgccctgccggatcaagcagattatcaatatgtggcaggaggtcggcaaggccatgtacgcccctcccatccggggccagatccggtgcagcagcaacatcaccggcctgctgctgacccgggacggcggcaacgataccagcggcaccgagatcttccggcctggcggcggagatatgcgggacaactggcggagcgagctgtacaagtacaaggtggtgaagatcgagcccctgggcgtggctcccaccaaggccaagcggcgggtggtgcagagcgagaagagcgccgtgggcatcggcgccgtgtttctgggcttcctgggagccgccggaagcaccatgggagccgccagcatgaccctgaccgtgcaggcccggctgctgctgtccggcatcgtgcagcagcagaacaacctgctccgggccatcgaggcccagcagcacctgctgcagctgaccgtgtggggcatcaagcagctgcaggccagggtgctggccgtggagagatacctgaaggatcagcagctcctggggatctggggctgcagcggcaagctgatctgcaccaccaccgtgccctggaacgccagctggtccaacaagagcctggacaagatctggaacaatatgacctggatggaatgggagcgcgagatcaacaattacaccagcctgatctacaccctgatcgaggaaagccagaaccagcaggaaaagaacgagcaggaactgctggaactggacaagtgggccagcctgtggaactggttcgacatcagcaactggctgtgg

SEQ ID NO: 6 - DNA, 2133 bp, Artificial Sequence
  Mos2SEnv mosaic antigen sequence
atgagagtgcggggcatgctgagaaactggcagcagtggtggatctggtccagcctgggcttctggatgctgatgatctacagcgtgatgggcaacctgtgggtcaccgtgtactacggcgtgcccgtgtggaaggacgccaagaccaccctgttttgcgcctccgatgccaaggcctacgagaaagaggtgcacaacgtctgggccacccacgcctgtgtgcccaccgaccccaatccccaggaaatcgtcctgggcaacgtgaccgagaacttcaacatgtggaagaacgacatggtcgatcagatgcacgaggacatcatctccctgtgggacgcctccctggaaccctgcgtgaagctgacccctctgtgcgtgaccctgaactgccggaacgtgcgcaacgtgtccagcaacggcacctacaacatcatccacaacgagacatacaaagagatgaagaactgcagcttcaacgctaccaccgtggtcgaggaccggaagcagaaggtgcacgccctgttctaccggctggacatcgtgcccctggacgagaacaacagcagcgagaagtcctccgagaacagctccgagtactacagactgatcaactgcaacaccagcgccatcacccaggcctgccccaaggtgtccttcgaccctatccccatccactactgcgcccctgccggctacgccatcctgaagtgcaacaacaagaccttcaatggcaccggcccctgcaacaatgtgtccaccgtgcagtgcacccacggcatcaagcccgtggtgtctacccagctgctgctgaacggcagcctggccgaggaagagatcattatcagaagcgagaacctgaccaacaacgccaaaaccatcatcgtccacctgaacgaaaccgtgaacatcacctgtacccggcctaacaacaacacccggaagtccatccggatcggccctggccagaccttttacgccaccggcgatattatcggcgacatccggcaggcccactgcaatctgagccgggacggctggaacaagacactgcagggcgtcaagaagaagctggccgaacacttccctaacaagactatcaagttcgcccctcactctggcggcgacctggaaatcaccacccacaccttcaactgtcggggcgagttcttctactgcaatacctccaacctgttcaacgagagcaacatcgagcggaacgacagcatcatcacactgccttgccggatcaagcagattatcaatatgtggcaggaagtgggcagagccatctacgcccctccaatcgccggcaacatcacatgccggtccaatatcaccggcctgctgctcaccagagatggcggctccaacaatggcgtgccaaacgacaccgagacattcagacccggcggaggcgacatgcggaacaattggcggagcgagctgtacaagtacaaggtggtggaagtgaagcccctgggcgtggcccctaccgaggccaagagaagagtggtcgaacgcgagaagcgggccgtgggaatcggagccgtgtttctgggaatcctgggagccgctggctctaccatgggcgctgcctctatcaccctgacagtgcaggccagacagctgctcagcggcatcgtgcagcagcagagcaacctgctgagagccattgaggcccagcagcacatgctgcagctgaccgtgtggggcattaagcagctccagacacgggtgctggccatcgagagatacctgcaggatcagcagctcctgggcctgtggggctgtagcggcaagctgatctgtaccaccgccgtgccctggaatacctcttggagcaacaagagccagaccgacatctgggacaacatgacctggatgcagtgggacaaagaaatcggcaactataccggcgagatctatagactgctggaagagtcccagaaccagcaggaaaagaacgagaaggacctgctggccctggattcttggaacaatctgtggaactggttcagcatctccaagtggctgtggtacatcaagatcttcatcatgatcgtgggcggcctgatcggcctgcggatcatctttgccgtgctgagcatcgtgaaccgcgtgcggcagggctac

SEQ ID NO: 7 - DNA, 4050 bp, Artificial Sequence
  Mos1.Gag-Pol mosaic antigen sequence
atgggagccagagccagcgtgctgtccggaggggagctggaccgctgggagaagatcaggctgaggcctggagggaagaagaagtacaggctgaagcacatcgtgtgggccagcagagagctggaacggtttgccgtgaaccctggcctgctggaaaccagcgagggctgtaggcagattctgggacagctgcagcccagcctgcagacaggcagcgaggaactgcggagcctgtacaacaccgtggccaccctgtactgcgtgcaccagcggatcgagatcaaggacaccaaagaagccctggaaaagatcgaggaagagcagaacaagagcaagaagaaagcccagcaggctgccgctgacacaggcaacagcagccaggtgtcccagaactaccccatcgtgcagaacatccagggacagatggtgcaccaggccatcagccctcggaccctgaacgcctgggtgaaggtggtggaggaaaaggccttcagccctgaggtgatccccatgttctctgccctgagcgagggagccacaccccaggacctgaacaccatgctgaacaccgtgggagggcaccaggctgccatgcagatgctgaaagagacaatcaacgaggaagctgccgagtgggacagggtccacccagtgcacgctggacctatcgctcctggccagatgagagagcccagaggcagcgatattgctggcaccacctccacactgcaggaacagatcggctggatgaccaacaaccctcccatccctgtgggagagatctacaagcggtggatcattctgggactgaacaagatcgtgcggatgtacagccctgtgagcatcctggacatcaggcagggacccaaagagcccttcagggactacgtggaccggttctacaagaccctgagagccgagcaggccagccaggacgtgaagaactggatgaccgagacactgctggtgcagaacgccaaccctgactgcaagaccatcctgaaagccctgggacctgctgccaccctggaagagatgatgacagcctgccagggagtgggaggacctggccacaaggccagggtgctggccgaggccatgagccaggtgaccaactctgccaccatcatgatgcagagaggcaacttccggaaccagagaaagaccgtgaagtgcttcaactgtggcaaagagggacacattgccaagaactgcagggctcccaggaagaaaggctgctggaagtgcggaaaagaaggccaccagatgaaggactgcaccgagaggcaggccaacttcctgggcaagatctggcctagcaacaagggcaggcctggcaacttcctgcagaacagacccgagcccaccgctcctcccgaggaaagcttccggtttggcgaggaaaccaccacccctagccagaagcaggaacccatcgacaaagagatgtaccctctggccagcctgaagagcctgttcggcaacgaccccagcagccagatggctcccatcagcccaatcgagacagtgcctgtgaagctgaagcctggcatggacggacccagggtgaagcagtggcctctgaccgaggaaaagatcaaagccctgacagccatctgcgaggaaatggaaaaagagggcaagatcaccaagatcggacccgagaacccctacaacacccctgtgttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggctgtgggagatgcctacttcagcgtgcctctggacgagggcttccggaagtacacagccttcaccatccccagcaccaacaacgagacacctggcatcagataccagtacaacgtgctgcctcagggctggaaaggcagccctgccatcttccagtgcagcatgaccagaatcctggaacccttcagagccaagaaccctgagatcgtgatctaccagtatatggctgccctctacgtgggcagcgacctggaaatcggacagcacagagccaaaatcgaagaactccgcgagcacctgctgaagtggggattcaccacccctgacaagaagcaccagaaagagcctcccttcctgtggatgggctacgagctgcaccctgacaagtggaccgtgcagcccatccagctgccagagaaggactcctggaccgtgaacgacatccagaaactggtcggcaagctgaactgggccagccagatctaccctggcatcaaagtcagacagctgtgtaagctgctgaggggagccaaagcactgaccgacatcgtgcctctgacagaagaagccgagctggaactggccgagaacagagagatcctgaaagaacccgtgcacggagtgtactacgacccctccaaggacctgattgccgagatccagaaacagggacacgaccagtggacctaccagatctatcaggaacctttcaagaacctgaaaacaggcaagtacgccaagatgcggacagcccacaccaacgacgtgaagcagctgaccgaagccgtgcagaaaatcgccatggaaagcatcgtgatctggggaaagacacccaagttcaggctgcccatccagaaagagacatgggaaacctggtggaccgactactggcaggccacctggattcccgagtgggagttcgtgaacaccccacccctggtgaagctgtggtatcagctggaaaaggaccctatcgctggcgtggagacattctacgtggctggagctgccaacagagagacaaagctgggcaaggctggctacgtgaccgacagaggcagacagaaaatcgtgagcctgaccgaaaccaccaaccagaaaacagccctgcaggccatctatctggcactgcaggacagcggaagcgaggtgaacatcgtgacagccagccagtatgccctgggcatcatccaggcccagcctgacaagagcgagagcgagctggtgaaccagatcatcgagcagctgatcaagaaagaacgggtgtacctgagctgggtgccagcccacaagggcatcggagggaacgagcaggtggacaagctggtgtccagcggaatccggaaggtgctgttcctggacggcatcgataaagcccaggaagagcacgagaagtaccacagcaattggagagccatggccagcgacttcaacctgcctcccgtggtggccaaagaaatcgtggccagctgcgaccagtgccagctgaaaggcgaggccatgcacggacaggtggactgctcccctggcatctggcagctggcatgcacccacctggaaggcaagatcattctggtggccgtgcacgtggccagcggatacatcgaagccgaagtgatccctgccgagacagggcaggaaacagcctacttcatcctgaagctggctggcagatggcctgtgaaggtgatccacacagccaacggcagcaacttcacctctgctgccgtgaaggctgcctgttggtgggctggcattcagcaggaatttggcatcccctacaatccccagtctcagggagtggtggccagcatgaacaaagagctgaagaagatcatcggacaggtcagggatcaggccgagcacctgaaaactgccgtccagatggccgtgttcatccacaacttcaagcggaagggagggatcggagggtactctgctggcgagcggatcatcgacatcattgccaccgatatccagaccaaagagctgcagaaacagatcatcaagatccagaacttcagggtgtactacagggacagcagggaccccatctggaagggacctgccaagctgctgtggaaaggcgaaggagccgtcgtcatccaggacaacagcgacatcaaggtggtgcccagacggaaggtgaaaatcatcaaggactacggcaaacagatggctggagccgactgtgtcgctggcaggcaggacgaggac

SEQ ID NO: 8 - DNA, 4023 bp, Artificial Sequence
  Mos2.Gag-Pol mosaic antigen sequence
atgggagccagagccagcatcctgcgaggagggaagctggacaagtgggagaagatcaggctgaggcctggagggaagaaacactacatgctgaagcacctggtctgggccagcagagagctggaacggtttgccctcaatcctggcctgctggaaaccagcgagggctgcaagcagatcatcaagcagctgcagcctgccctgcagacaggcaccgaggaactgcggagcctgttcaacaccgtggccaccctgtactgcgtgcatgccgagatcgaagtgagggacaccaaagaagccctggacaagatcgaggaagagcagaacaagagccagcagaaaacccagcaggccaaagaagccgacggcaaggtctcccagaactaccccatcgtgcagaacctgcagggacagatggtgcaccagcccatcagccctcggacactgaatgcctgggtgaaggtgatcgaggaaaaggccttcagccctgaggtgatccccatgttcacagccctgagcgagggagccacaccccaggacctgaacaccatgctgaacaccgtgggagggcaccaggctgccatgcagatgctgaaggacaccatcaacgaggaagctgccgagtgggacaggctgcaccctgtgcacgctggacctgtggctcctggccagatgagagagcccagaggcagcgatattgctggcaccacctccaatctgcaggaacagatcgcctggatgaccagcaaccctcccatccctgtgggagacatctacaagcggtggatcatcctgggactgaacaagatcgtgcggatgtacagccctacctccatcctggacatcaagcagggacccaaagagcctttcagggactacgtggaccggttcttcaagaccctgagagccgagcaggccacccaggacgtgaagaactggatgaccgacaccctgctggtgcagaacgccaaccctgactgcaagaccatcctgagagccctgggacctggagccaccctggaagagatgatgacagcctgccagggagtgggaggaccctctcacaaggctagggtgctggccgaggccatgagccagaccaacagcaccatcctgatgcagcggagcaacttcaagggcagcaagcggatcgtgaagtgcttcaactgtggcaaagagggacacattgccagaaactgtagggcacccaggaagaaaggctgctggaagtgcggaaaagaaggccaccagatgaaggactgcaccgagaggcaggccaacttcctgggcaagatctggcctagccacaagggcagacctggcaacttcctgcagagcagacccgagcccaccgctcctccagccgagagcttccggttcgaggaaaccacccctgctcccaagcaggaacctaaggacagagagcctctgaccagcctgagaagcctgttcggcagcgaccctctgagccagatggctcccatctcccctatcgagacagtgcctgtgaagctgaagcctggcatggacggacccaaggtgaaacagtggcctctgaccgaggaaaagatcaaagccctggtggagatctgtaccgagatggaaaaagagggcaagatcagcaagatcggacccgagaacccctacaacacccctatcttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggccgtgggagatgcctacttcagcgtgcctctggacgaggacttcagaaagtacacagccttcaccatccccagcatcaacaacgagacacctggcatcagataccagtacaacgtgctgcctcagggatggaagggctctcctgcaatcttccagagcagcatgaccaagatcctggaacccttccggaagcagaaccctgacatcgtgatctaccagtacatggcagccctgtacgtcggcagcgacctggaaatcggacagcaccggaccaagatcgaagaactcaggcagcacctgctgcggtggggattcaccacccctgacaagaagcaccagaaagagcctcccttcctgtggatgggctacgagctgcacccagacaagtggaccgtgcagcccatcgtgctgcctgagaaggactcctggaccgtgaacgacatccagaaactggtcggcaagctgaactgggccagccagatctacgctggcatcaaagtgaagcagctgtgtaagctcctgagaggcaccaaagccctgaccgaggtggtgccactgacagaggaagccgagctggaactggccgagaacagagagatcctgaaagaacccgtgcacggagtgtactacgaccccagcaaggacctgattgccgagatccagaagcagggacagggacagtggacctaccagatctaccaggaacccttcaagaacctgaaaacaggcaagtacgccaggatgaggggagcccacaccaacgacgtcaaacagctgaccgaagccgtgcagaagatcgccaccgagagcatcgtgatttggggaaagacacccaagttcaagctgcccatccagaaagagacatgggaggcctggtggaccgagtactggcaggccacctggattcccgagtgggagttcgtgaacaccccacccctggtgaagctgtggtatcagctggaaaaagaacccatcgtgggagccgagacattctacgtggctggagctgccaacagagagacaaagctgggcaaggctggctacgtgaccgacagaggcaggcagaaagtggtgtccctgaccgataccaccaaccagaaaacagccctgcaggccatccacctggctctgcaggactctggcctggaagtgaacatcgtgacagccagccagtatgccctgggcatcattcaggcacagcctgacaagagcgagagcgagctggtgtctcagatcattgagcagctgatcaagaaagaaaaggtgtacctggcctgggtgccagcccacaaggggatcggagggaacgagcaggtggacaagctggtgtccaggggcatccggaaggtgctgtttctggacggcatcgacaaagcccaggaagagcacgagaagtaccacagcaattggagagccatggccagcgagttcaacctgcctcccatcgtggccaaagaaatcgtggcctcttgcgacaagtgccagctgaaaggcgaggccattcacggacaggtggactgcagcccaggcatctggcagctggcctgcacccacctggaaggcaaggtgatcctggtggccgtgcacgtggcctctggatacatcgaagccgaagtgatccctgccgagacaggccaggaaacagcctacttcctgctgaagctggctggcaggtggcctgtgaaaaccatccacacagccaacggcagcaacttcacctctgccaccgtgaaggctgcctgttggtgggctggcattaagcaggaatttggcatcccctacaaccctcagtctcagggagtggtggcctccatcaacaaagagctgaagaagatcatcggacaggtcagggatcaggccgagcatctgaaaacagccgtccagatggccgtgttcatccacaacttcaagcggaagggagggatcggagagtactctgctggcgagaggatcgtggacattatcgccagcgatatccagaccaaagaactgcagaagcagatcacaaagatccagaacttcagggtgtactacagggacagcagagatcccctgtggaagggacctgccaagctgctgtggaaaggcgaaggagccgtcgtcatccaggacaacagcgacatcaaggtggtgcccagacggaaggccaagatcatcagagactacggcaaacagatggctggcgacgactgcgtcgcctctaggcaggacgaggac

SEQ ID NO: 9 - protein, 132 aa, Artificial Sequence
  PGT-121 variable heavy chain
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 10 - protein, 105 aa, Artificial Sequence
  PGT-121 variable light chain
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 11 - protein, 462 aa, Artificial Sequence
  PGT-121 heavy chain
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 211 aa, Artificial Sequence
  PGT-121 light chain
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 13 - protein, 141 aa, Artificial Sequence
  PGDM1400 variable heavy chain
QVHLTQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSS

SEQ ID NO: 14 - protein, 112 aa, Artificial Sequence
  PGDM1400 variable light chain
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIK

SEQ ID NO: 15 - protein, 471 aa, Artificial Sequence
  PGDM1400 heavy chain
QVHLTQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 219 aa, Artificial Sequence
  PGDM1400 light chain
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 125 aa, Artificial Sequence
  VRC07-523LS variable heavy chain
QVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSS

SEQ ID NO: 18 - protein, 101 aa, Artificial Sequence
  VRC07-523LS variable light chain
SLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 19 - protein, 455 aa, Artificial Sequence
  VRC07-523LS heavy chain
QVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 20 - protein, 208 aa, Artificial Sequence
  VRC07-523LS light chain
SLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0000973 A1
(43) Pub. Date: Jan. 5, 2023

(54) Combinations of Vaccines and Neutralizing Antibodies for Treating Human Immunodeficiency Virus Infection in Subjects Undergoing Antiretroviral Treatment

(71) Applicants: Janssen Vaccines & Prevention B.V., Leiden (NL); Beth Israel Deaconess Medical Center, Inc., Boston, MA (US)

(72) Inventors: Dan H. Barouch, Newton, MA (US); Boris D. Juelg, Winchester, MA (US); Maria Grazia Pau, Leiden (NL); Frank Tomaka, Titusville, NJ (US)

(21) Appl. No.: 17/808,348

(22) Filed: Jun. 23, 2022

Related U.S. Application Data
(60) Provisional application No. 63/202,808, filed on Jun. 25, 2021.

(51) Int. Cl. A61K 39/21; A61K 39/42; A61K 9/00; A61P 31/18; A61P 37/04

(52) U.S. Cl. CPC A61K 39/21; A61K 39/42; A61K 9/0019; A61P 31/18; A61P 37/04; A61K 2039/5256

(57) ABSTRACT

Methods for inducing an immune response against Human Immunodeficiency Virus (HIV) in HIV-infected subjects undergoing antiretroviral therapy (ART) are described. The methods involve initial administration of an adenovirus vector vaccine and subsequent administration of a poxvirus vector vaccine, followed by administration of anti-HIV broadly neutralizing antibodies (bNAb).

### CROSS REFERENCE TO RELATED APPLICATION

This application claims the benefit of U.S. Provisional Patent Application No. 62/202,808, filed Jun. 25, 2021, the disclosures of which is incorporated by reference herein.

### STATEMENT REGARDING FEDERALLY SPONSORED RESEARCH OR DEVELOPMENT

This invention was made with government support under Grant No. AI145801 awarded by the National Institutes of Health. The government has certain rights in the invention

### REFERENCE TO SEQUENCE LISTING SUBMITTED ELECTRONICALLY

This application contains a sequence listing that is submitted electronically via EFS-Web as an ASCII formatted sequence listing with a file name “065768-100US2 Sequence Listing” and a creation date of Jun. 15, 2022, and having a size of 76 kb. The sequence listing is submitted via EFS-Web is part of the specification and is herein incorporated by reference in its entirety.

### BACKGROUND

The number of new HIV infections and the number of acquired immunodeficiency syndrome (AIDS) related deaths are declining. Nevertheless, globally, an estimated 38 million people were living with human immunodeficiency virus (HIV) in 2019 (www.unaids.org/en/resources/fact-sheet), which is an increase from previous years as a result of the wider availability of life-saving antiretroviral therapies (ART).

Despite its proven success at suppressing viral replication saving lives, there are significant challenges to initiating and maintaining ART for all of those HIV-infected patients that need it in the world. For example, ART does not eliminate the viral reservoir, and treatment is associated with an incomplete restoration of the host immune system. In particular, while ART facilitates CD4⁺ T cell reconstitution in the blood, there is only a limited improvement in the function of anti-HIV specific CD8⁺ T cell responses. Also, ART must be taken life-long with near perfect adherence in order to be effective. This places extreme pressure and costs on international donors and over-taxed health systems in developing countries where HIV prevalence rates are highest. Moreover, ART has both short-term and long-term side effects for users, and drug resistance rates rise as more people are on treatment for longer periods of time. Thus, alternative or complementary treatments, including a therapeutic vaccine, which could induce a true or “functional” cure of HIV infection and lessen or eliminate the need for lifelong ART for HIV infected individuals, would therefore be of great benefit. The concept of a “functional cure” includes therapeutic strategies that enable host control of the virus without the need for antiretroviral treatment.

Accordingly, there is a need for improved methods of treating HIV-infected subjects, particularly novel therapies that seek to achieve a functional cure of HIV, including a therapeutic vaccine that preferably would improve immune responses to HIV and possibly allow at least some treated subjects to discontinue ART while maintaining viremic control.

### BRIEF SUMMARY

The application relates to methods for inducing an immune response against human immunodeficiency virus (HIV) in an HIV-infected subject undergoing antiretroviral therapy (ART) with a first vaccine comprising one or more adenovirus vectors encoding mosaic HIV immunogens, a second vaccine comprising poxvirus vector(s) encoding the mosaic HIV immunogens, and at least two, preferably three different anti-HIV broadly neutralizing antibodies (bNAbs).

In one general aspect, the application relates to a method of inducing an immune response against a human immunodeficiency virus (HIV) in an HIV-infected human subject undergoing antiretroviral therapy (ART), the method comprising:

(i) administering to the human subject an adenovirus vaccine comprising one or more adenovirus vectors together encoding HIV gag, pol, and env immunogens, preferably four HIV immunogens having the amino acid sequences of SEQ ID NO: 1, SEQ ID NO: 2, SEQ ID NO: 3, and SEQ ID NO: 4, and a pharmaceutically acceptable carrier;

(ii) administering to the human subject a poxvirus vaccine comprising one or more poxvirus vectors together encoding the HIV gag, pol, and env immunogens, preferably, the four HIV immunogens and a pharmaceutically acceptable carrier; and

(iii) administering to the human subject at least two, preferably three different anti-HIV broadly neutralizing antibodies (bNAbs) and a pharmaceutically acceptable carrier.

In certain embodiments, the one or more adenovirus vectors are adenovirus 26 (Ad26) vectors.

In certain embodiments, the adenovirus vaccine comprises more than one Ad26 vectors.

In certain embodiments, wherein the one or more poxvirus vectors are Modified Vaccinia Ankara (MVA) vectors.

In certain embodiments, the poxvirus vaccine consists of a single MVA vector encoding the four HIV immunogens.

In certain embodiments, the at least two anti-HIV bNAbs each bind to an epitope or region of HIV gp120 envelope selected from the group consisting of (i) CD4-binding site (CD4bs) (ii) third variable loop (V3) and/or high mannose patch comprising a N332 oligomannose glycan; (iii) second variable loop (V2) and/or Env trimer apex; (iv) gp120/gp41 interface; or (v) silent face of gp120. In certain embodiments, the at least two anti-HIV bNAbs comprise an anti-HIV bNAb binding to a CD4bs of HIV envelope protein, an anti-HIV bNAb binding to V3 of HIV envelope protein (preferably a glycan-dependent epitope therein), and an anti-HIV bNAb binding to V2 of HIV envelope protein.

In certain embodiments, the two or more anti-HIV bNAbs are selected from the group consisting of RC01, 3BNC117, VRC01-LS, VRC07-523LS, 10-1074, PGT121, and PGDM1400, preferably the two or more anti-HIV bNAbs are three anti-HIV bNAbs consisting of PGT121, PGDM1400, and VRC07-523LS.

In certain embodiments, the one or more adenovirus vectors together are administered at a total dose of about 5×10⁹ to about 1×10¹¹ viral particles (vp), preferably about 5×10¹⁰ vp, of the one or more adenovirus vectors in total, per administration.

In certain embodiments, the one or more poxvirus vectors together are administered at a total dose of about 1×10⁷ to about 5×10⁸ infectious units (IU), preferably about 2×10⁸ IU, of the one or more poxvirus vectors in total, per administration.

In certain embodiments, the poxvirus vaccine is administered 8-14 weeks, preferably 12 weeks, after the adenovirus vaccine is initially administered.

In certain embodiments, each anti-HIV bNAb is administered at a dose of about 5-40 mg/kg of the anti-HIV bNAb, preferably about 10-20 mg/kg of each anti-HIV bNAb, per administration.

In certain embodiments, the at least two, preferably three anti-HIV bNAbs are administered 20-30 weeks, preferably 24 weeks after the adenovirus vaccine is initially administered.

In certain embodiments, the two or more anti-HIV bNAbs are administered one or two times at about 20-30 weeks, such as at about 20, 21, 22, 23, 24, 25, 26, 27, 28, 29 or 30 weeks, preferably at about 24 and about 28 weeks, after the adenovirus vaccine is initially administered.

Also provided is a method of inducing an immune response against human immunodeficiency virus (HIV) in an HIV-infected human subject undergoing antiretroviral therapy (ART), the method comprising:

(i) intramuscularly administering to the human subject an adenovirus 26 (Ad26) vaccine comprising one or more Ad26 vectors together encoding four HIV immunogens having the amino acid sequences of SEQ ID NO: 1, SEQ ID NO: 2, SEQ ID NO: 3, and SEQ ID NO: 4, and a pharmaceutically acceptable carrier, in a total dose of about 5×10⁹ to about 1×10¹¹ viral particles (vp), preferably about 5×10¹⁰ vp, of the Ad26 vectors;

(ii) intramuscularly administering to the human subject an Modified Vaccinia Ankara (MVA) vaccine comprising one or more MVA vectors, preferably one MVA vector, encoding the four HIV immunogens, and a pharmaceutically acceptable carrier, in a total dose of about 1×10⁷ to about 5×10⁸ infectious units (IU), preferably about 2×10⁸ IU, of the one or more MVA vectors, wherein the MVA vaccine, is administered 8-14 weeks, preferably 12 weeks, after the Ad26 vaccine is initially administered in step (i); and

(iii) intravenously administering to the human subject PGT121, PGDM1400, and VRC07-523LS anti-HIV broadly neutralizing antibodies (bNAbs) and one or more pharmaceutically acceptable carriers, at a dose of about 5 mg/kg to about 40 mg/kg of each anti-HIV bNAb, per administration, preferably the anti-HIV bNAbs are administered one or two times at 20-30 weeks after the Ad26 vaccine is initially administered in step (i); more preferably, at a dose of 10-20 mg/kg of each anti-HIV bNAb per administration at 24 and 28 weeks after the Ad26 vaccine is initially administered in step (i).

In certain embodiments, PGT121 is administered at a dose of 20 mg/kg of PGT121, per administration, at 24 and 28 weeks after the Ad26 vaccine is initially administered in step (i).

In certain embodiments, PGDM1400 is administered at a dose of 20 mg/kg of PGDM1400, per administration, at 24 and 28 weeks after the Ad26 vaccine is initially administered in step (i).

In certain embodiments, VRC07-523LS is administered at a dose of 10 mg/kg of VRC07-523LS at 24 weeks after the Ad26 vaccine is initially administered in step (i).

In certain embodiments, the human subject has undergone ART for at least 48 weeks prior to being initially administered the adenovirus vaccine.

In certain embodiments, the human subject continues undergoing suppressive ART during the treatment.

In certain embodiments, the suppressive ART is stopped after the initial administration of the at least two anti-HIV bNAbs.

Also provided is a method of treating a human immunodeficiency virus (HIV) infection in a human subject in need thereof, comprising:

(i) treating the human subject with an antiretroviral therapy (ART); and

(ii) inducing an immune response against the HIV in the human subject using a method of the application.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the application described herein. Such equivalents are intended to be encompassed by the application.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “subject” means a human, who will be or has been treated by a method according to an embodiment of the application.

As used herein, the terms and phrases “in combination,” “in combination with,” “co-delivery,” and “administered together with” in the context of the administration of two or more therapies or components to a subject refers to simultaneous administration of two or more therapies or components. “Simultaneous administration” can be administration of the two components at least within the same day. When two components are “administered together with” or “administered in combination with,” they can be administered in separate compositions sequentially within a short time period, such as 24, 20, 16, 12, 8 or 4 hours, or within 1 hour, or within 30 minutes, or within 10 minutes, or within 5 minutes, or within 2 minutes, or they can be administered in a single composition at the same time. The use of the term “in combination with” does not restrict the order in which therapies or components are administered to a subject. For example, a first therapy or component can be administered prior to (e.g., 5 minutes to one hour before), concomitantly with or simultaneously with, or subsequent to (e.g., 5 minutes to one hour after) the administration of a second therapy.

The application relates to methods of inducing an immune response against human immunodeficiency virus (HIV) in an HIV-infected human subject undergoing antiretroviral treatment (ART). According to embodiments of the application, an adenovirus vaccine comprises one or more adenovirus vectors encoding HIV gag, pol, and env immunogens, preferably four HIV immunogens having the amino acid sequences of SEQ ID NO: 1, SEQ ID NO: 2, SEQ ID NO: 3, and SEQ ID NO: 4. According to the application, a poxvirus vaccine comprises one or more poxvirus vectors, preferably one or more Modified Vaccinia Ankara (MVA) vectors, encoding the four HIV immunogens, i.e. HIV immunogens having the amino acid sequences of SEQ ID NO: 1, SEQ ID NO: 2, SEQ ID NO: 3, and SEQ ID NO: 4.

### Human Immunodeficiency Virus (HIV)

Human immunodeficiency virus (HIV) is a member of the genus Lentivirinae, which is part of the family of Retroviridae. Two species of HIV infect humans: HIV-1 and HIV-2. HIV-1 is the most common strain of HIV virus and is known to be more pathogenic than HIV-2. As used herein, the terms “human immunodeficiency virus” and “HIV” refer, but are not limited to, HIV-1 and HIV-2. In preferred embodiments, HIV refers to HIV-1.

HIV is categorized into multiple clades with a high degree of genetic divergence. As used herein, the term “HIV clade” or “HIV subtype” refers to related human immunodeficiency viruses classified according to their degree of genetic similarity. There are currently three groups of HIV-1 isolates: M, N and O. Group M (major strains) consists of at least ten clades, A through J. Group O (outer strains) can consist of a similar number of clades. Group N is a new HIV-1 isolate that has not been categorized in either group M or O.

According to embodiments of the application, the methods described herein can be used to induce an immune response against one or more clades of HIV.

### HIV Immunogens

As used herein, the terms “HIV antigen,” “antigenic polypeptide of an HIV,” “HIV antigenic polypeptide,” “HIV antigenic protein,” “HIV immunogenic polypeptide,” and “HIV immunogen” all refer to a polypeptide capable of inducing an immune response, e.g., a humoral and/or cellular mediated response, against HIV in a subject. The HIV immunogen can be a protein of HIV, a fragment or epitope thereof, or a combination of multiple HIV proteins or portions thereof, that can induce an immune response against HIV in a subject. An HIV immunogen is capable of raising in a host a protective immune response, e.g., inducing an immune response against a viral disease or infection, and/or producing an immunity in (i.e., vaccinates) a subject against a viral disease or infection, that protects the subject against the viral disease or infection. For example, the HIV immunogen can comprise a protein or fragment(s) thereof from HIV, such as the HIV gag, pol and env gene products.

According to embodiments of the application, the HIV immunogen can be an HIV-1 or HIV-2 immunogen or fragment(s) thereof. Examples of HIV immunogens include, but are not limited to gag, pol, and env gene products, which encode structural proteins and essential enzymes. Gag, pol, and env gene products are synthesized as polyproteins, which are further processed into multiple other protein products. The primary protein product of the gag gene is the viral structural protein gag polyprotein, which is further processed into MA, CA, SP1, NC, SP2, and P6 protein products. The pol gene encodes viral enzymes (Pol, polymerase), and the primary protein product is further processed into RT, RNase H, IN, and PR protein products. The env gene encodes structural proteins, specifically glycoproteins of the virion envelope. The primary protein product of the env gene is gp160, which is further processed into gp120 and gp41. A heterologous gene according to the application preferably encodes a gag, env, and/or pol gene product, or portion thereof. According to a preferred embodiment, the HIV immunogen comprises an HIV Gag, Env, or Pol immunogen, or any portion or combination thereof, more preferably an HIV-1 Gag, Env, or Pol immunogen, or any portion or combination thereof. The heterologous gene(s) encoding the HIV immunogen(s) preferably are encoded in an adenovirus vector, such as an Ad26 vector, and in a poxvirus vector, such as an MVA vector.

According to preferred embodiments of the application, an HIV immunogen is a mosaic HIV immunogen. As used herein, “mosaic immunogen” refers to a recombinant protein assembled from fragments of natural sequences. The “mosaic immunogen” can be computationally generated and optimized using a genetic algorithm. Mosaic immunogens resemble natural antigens, but are optimized to maximize the coverage of potential T-cell epitopes found in the natural sequences, which improves the breadth and coverage of the immune response.

Examples of mosaic HIV Gag-Pol-Env immunogens include those described in, e.g., US20120076812, Barouch et al., Nat Med 2010, 16:319-323; Barouch et al., Cell 155:1-9, 2013; and WO 2017/102929, all of which are incorporated herein by reference in their entirety.

Preferably, the mosaic HIV immunogens encoded by the vectors according to the application comprise one or more of the amino acid sequences selected from the group consisting of SEQ ID NOs: 1-4. Alternative and/or additional HIV immunogens could be encoded by each or either of the vaccine vectors of the application in certain embodiments, e.g. to further broaden the immune response.

In view of the present disclosure, a mosaic HIV immunogen can be produced using methods known in the art. See, e.g., US20120076812, Fischer et al, Nat Med, 2007. 13(1): p. 100-6; Barouch et al., Nat Med 2010, 16:319-323, all of which are incorporated herein by reference in their entirety.

### Adenovirus Vector

Vaccines used in the initial administration step (sometimes referred to as ‘priming’ step) of the methods of the application comprise one or more adenovirus vectors encoding one more mosaic HIV immunogens.

An adenovirus according to the application belongs to the family of the Adenoviridae, and preferably is one that belongs to the genus Mastadenovirus. It can be a human adenovirus, but also an adenovirus that infects other species, including but not limited to a bovine adenovirus (e.g. bovine adenovirus 3, BAdV3), a canine adenovirus (e.g. CAdV2), a porcine adenovirus (e.g. PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu), or a simian adenovirus such as chimpanzee or gorilla adenovirus (ChAd, AdCh, or SAdV), or a rhesus monkey adenovirus (RhAd). In the invention, a human adenovirus is meant if referred to as Ad without indication of species, e.g., the brief notation “Ad26” means the same as HAdV26, which is human adenovirus serotype 26. Also as used herein, the notation “rAd” means recombinant adenovirus, e.g., “rAd26” refers to recombinant human adenovirus 26.

Most advanced studies have been performed using human adenoviruses, and human adenoviruses are preferred according to certain aspects of the invention. In certain preferred embodiments, a recombinant adenovirus according to the invention is based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26. Advantages of these serotypes include a low seroprevalence and/or generally low pre-existing neutralizing antibody titers in the human population, induction of good immune responses against the immunogens encoded in such vectors, and experience with use in human subjects.

Simian adenoviruses generally also have a low seroprevalence and/or low pre-existing neutralizing antibody titers in the human population, and a significant amount of work has been reported using chimpanzee adenovirus vectors (e.g., U.S. Pat. No. 6,083,716; WO 2005/071093; WO 2010/086189; WO 2010085984). Hence, in other embodiments, the recombinant adenovirus according to the invention is based upon a simian adenovirus, e.g., a chimpanzee adenovirus. In certain embodiments, the recombinant adenovirus is based upon simian adenovirus type 1, 7, 8, 21, 22, 23, 24, 25, 26, 27.1, 28.1, 29, 30, 31.1, 32, 33, 34, 35.1, 36, 37.2, 39, 40.1, 41.1, 42.1, 43, 44, 45, 46, 48, 49, 50 or SA7P. In certain embodiments, the recombinant adenovirus is based upon a chimpanzee adenovirus such as ChAdOx 1 (see e.g., WO 2012/172277), or ChAdOx 2 (see e.g., WO 2018/215766). In certain embodiments, the recombinant adenovirus is based upon a chimpanzee adenovirus such as BZ28 (see e.g., WO 2019/086466). In certain embodiments, the recombinant adenovirus is based upon a gorilla adenovirus such as BLY6 (see e.g., WO 2019/086456), or BZ1 (see e.g. WO 2019/086466).

Preferably, the adenovirus vector is a replication deficient recombinant viral vector, such as rAd26, rAd35, rAd48, rAd5HVR48, etc.

In a preferred embodiment of the invention, the adenoviral vectors comprise capsid proteins from rare serotypes, e.g., including Ad26. In the typical embodiment, the vector is an rAd26 virus. An “adenovirus capsid protein” refers to a protein on the capsid of an adenovirus (e.g., Ad26, Ad35, rAd48, rAd5HVR48 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton and fiber are of Ad26.

One of ordinary skill in the art will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the invention could combine the absence of pre-existing immunity of a first serotype with characteristics such as temperature stability, assembly, anchoring, production yield, redirected or improved infection, stability of the DNA in the target cell, and the like. See for example WO 2006/040330 for chimeric adenovirus Ad5HVR48, that includes an Ad5 backbone having partial capsids from Ad48, and also e.g., WO 2019/086461 for chimeric adenoviruses Ad26HVRPtr1, Ad26HVRPtr12, and Ad26HVRPtr13, that include an Ad26 virus backbone having partial capsid proteins of Ptr1, Ptr12, and Ptr13, respectively).

According to embodiments of the application, an adenovirus is a human adenovirus serotype 26 (Ad26). An advantage of human adenovirus serotype 26 is that, so far, significant experience was obtained with such vectors in humans, and this did not reveal that pre-existing neutralizing antibody responses against such vectors would cause substantial interference with desired vaccine-induced responses, e.g., against the encoded immunogens in such vectors. Preferably, the adenovirus vector is a replication deficient recombinant viral vector, such as a replication deficient recombinant adenovirus 26 vector.

In certain embodiments, the recombinant adenovirus vector useful in the application is derived mainly or entirely from Ad26 (i.e., the vector is rAd26). In some embodiments, the adenovirus is replication deficient, e.g., because it contains a deletion in the E1 region of the genome. For the adenoviruses derived from Ad26 used in the application, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g., Havenga, et al., 2006, J Gen Virol 87: 2135-43; WO 03/104467). However, such adenoviruses will not be capable of replicating in non-complementing cells that do not express the E1 genes of Ad5. Thus, in certain embodiments, the adenovirus is a human adenovirus of serotype 26, with a deletion in the E1 region into which the nucleic acid encoding one or more mosaic HIV immunogens has been cloned, and with an E4 orf6 region of Ad5.

The preparation of recombinant adenoviral vectors is well known in the art. Preparation of rAd26 vectors is described, for example, in WO 2007/104792 and in Abbink et al., (2007) Virol 81(9): 4654-63, both of which are incorporated by reference herein in their entirety. Exemplary genome sequences of Ad26 are found in GenBank Accession EF 153474 and in SEQ ID NO: 1 of WO 2007/104792, which is herein incorporated by reference in its entirety. Typically, an adenovirus vector useful in the application is produced using a nucleic acid comprising the entire recombinant adenoviral genome (e.g., a plasmid, cosmid, or baculovirus vector).

The adenovirus vectors useful in the application are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding an HIV immunogen (usually linked to a promoter) within the region. In some embodiments, the vectors of the application can contain deletions in other regions, such as the E3 region, or insertions of heterologous genes linked to a promoter within such regions. Mutations in the E3 region of the adenovirus need not be complemented by the cell line, since E3 is not required for replication.

A packaging cell line is typically used to produce sufficient amounts of adenovirus vectors for use in methods of the application. A packaging cell is a cell that comprises those genes that have been deleted or inactivated in a replication deficient vector, thus allowing the virus to replicate in the cell. Suitable packaging cell lines include, for example, PER.C6, 911, and HEK293.

According to embodiments of the application, HIV immunogens can be expressed in the adenovirus 26 vectors described herein. Optionally, the heterologous gene encoding the HIV immunogen can be codon-optimized to ensure proper expression in the treated host (e.g., human). Codon-optimization is a technology widely applied in the art. Typically, the heterologous gene encoding the HIV immunogen is cloned into the E1 and/or the E3 region of the adenoviral genome. Non-limiting embodiments of codon optimized nucleotide sequences encoding HIV immunogens with SEQ ID NOs: 1-4 are provided herein as SEQ ID NOs: 5-8, respectively.

According to embodiments of the application, one or more adenovirus vectors comprise nucleic acid that encodes one or more HIV immunogens, in particular the one or more Ad26 vectors together encode four mosaic HIV immunogens having the amino acid sequences of SEQ ID NO: 1, SEQ ID NO: 2, SEQ ID NO: 3, and SEQ ID NO: 4. In certain embodiments, the Ad26 vaccine used in the methods of the application comprises a first Ad26 vector encoding the HIV immunogen of SEQ ID NO: 1, a second Ad26 vector encoding the HIV immunogen of SEQ ID NO: 2, a third Ad26 vector encoding the HIV immunogen of SEQ ID NO: 3, and a fourth Ad26 vector encoding the HIV immunogen of SEQ ID NO: 4. In certain embodiments these vectors are present in a single composition in a 1:1:1:1 ratio (based on viral particles).

The heterologous gene encoding the mosaic HIV immunogen can be under the control of (i.e., operably linked to) an adenovirus-derived promoter (e.g., the Major Late Promoter), or can be under the control of a heterologous promoter. Examples of suitable heterologous promoters include the cytomegalovirus immediate early (CMV) promoter and the Rous sarcoma virus (RSV) promoter. Preferably, the promoter is located upstream of the heterologous gene encoding the mosaic HIV immunogen within an expression cassette. In a preferred embodiment, the heterologous promoter is a CMV promoter.

### Poxvirus Vectors

Vaccines used in the second administration step (sometimes referred to as ‘boosting’ step) of the methods of the application comprise one or more poxvirus vectors encoding one more HIV immunogens, e.g., mosaic HIV immunogens, for instance mosaic HIV gag, pol, and/or env immunogens. A poxvirus vector used in the methods of the application may be derived, for example, from vaccinia virus or modified vaccinia virus Ankara (MVA). In preferred embodiments, a poxvirus vector is derived from modified vaccinia virus Ankara (MVA).

In some embodiments, an MVA vaccine used in the methods of the application comprises one or more Modified Vaccinia Ankara (MVA) vectors together encoding four mosaic HIV immunogens, in particular the HIV immunogens of SEQ ID NO: 1, SEQ ID NO: 2, SEQ ID NO: 3, and SEQ ID NO: 4. MVA vectors useful in the methods of the application utilize attenuated virus derived from MVA virus, which is characterized by the loss of their capabilities to reproductively replicate in human cell lines.

MVA has been generated by more than 570 serial passages on chicken embryo fibroblasts of the dermal vaccinia strain Ankara (Chorioallantois vaccinia virus Ankara virus, CVA; for review see Mayr et al. (1975) Infection 3, 6-14) that was maintained in the Vaccination Institute, Ankara, Turkey for many years and used as the basis for vaccination of humans. In 1976, MVA derived from MVA-571 seed stock (corresponding to the 571st passage) was registered in Germany as the primer vaccine in a two-stage parenteral smallpox vaccination program. As a result of the passaging used to attenuate MVA, there are a number of different strains or isolates, depending on the number of passages conducted in CEF cells. For example, MVA-572 was used in a small dose as a pre-vaccine in Germany during the smallpox eradication program, and MVA-575 was extensively used as a veterinary vaccine. MVA as well as MVA-BN lacks approximately 15% (31 kb from six regions) of the genome compared with ancestral CVA virus. The deletions affect a number of virulence and host range genes, as well as the gene for Type A inclusion bodies. MVA-575 was deposited on Dec. 7, 2000, at the European Collection of Animal Cell Cultures (ECACC) under Accession No. V00120707.

Strains of MVA having enhanced safety profiles for the development of safer products, such as vaccines or pharmaceuticals, have been developed, for example by Bavarian Nordic. MVA was further passaged by Bavarian Nordic and is designated MVA-BN. A representative sample of MVA-BN was deposited on Aug. 30, 2000 at the European Collection of Cell Cultures (ECACC) under Accession No. V00083008. MVA-BN is further described in WO 02/42480 (US 2003/0206926) and WO 03/048184 (US 2006/0159699), both of which are incorporated by reference herein in their entirety.

“Derivatives” or “variants” of MVA refer to viruses exhibiting essentially the same replication characteristics as MVA as described herein, but exhibiting differences in one or more parts of their genomes. For example, MVA-BN as well as a derivative or variant of MVA-BN fails to reproductively replicate in vivo in humans and mice, even in severely immune suppressed mice. More specifically, MVA-BN or a derivative or variant of MVA-BN has preferably also the capability of reproductive replication in chicken embryo fibroblasts (CEF), but no capability of reproductive replication in the human keratinocyte cell line HaCat (Boukamp et al (1988), J Cell Biol. 106: 761-771), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, a derivative or variant of MVA-BN has a virus amplification ratio at least two fold less, more preferably three-fold less than MVA-575 in Hela cells and HaCaT cell lines. Tests and assays for these properties of MVA variants are described in WO 02/42480 (US 2003/0206926) and WO 03/048184 (US 2006/0159699).

The term “not capable of reproductive replication” or “no capability of reproductive replication” is, for example, described in WO 02/42480, which also teaches how to obtain MVA having the desired properties as mentioned above. The term applies to a virus that has a virus amplification ratio at 4 days after infection of less than 1 using the assays described in WO 02/42480 or in U.S. Pat. No. 6,761,893, both of which are incorporated by reference herein in their entirety.

The term “fails to reproductively replicate” refers to a virus that has a virus amplification ratio at 4 days after infection of less than 1. Assays described in WO 02/42480 or in U.S. Pat. No. 6,761,893 are applicable for the determination of the virus amplification ratio.

The advantages of MVA-based vaccine include their safety profile as well as availability for large scale vaccine production. Furthermore, in addition to its efficacy, the feasibility of industrial scale manufacturing can be beneficial. Additionally, MVA-based vaccines can deliver multiple heterologous immunogens and allow for simultaneous induction of humoral and cellular immunity.

MVA vectors useful for methods of the application can be prepared using methods known in the art, such as those described in WO/2002/042480, WO/2002/24224, US20110159036, U.S. Pat. No. 8,197,825, etc., the relevant disclosures of which are incorporated herein by reference.

In another aspect, replication deficient MVA viral strains can also be suitable for use in methods of the application, such as strains MVA-572 and MVA-575, or any other similarly attenuated MVA strain. Also suitable can be a mutant MVA, such as the deleted chorioallantois vaccinia virus Ankara (dCVA). A dCVA comprises del I, del II, del III, del IV, del V, and del VI deletion sites of the MVA genome. The sites are particularly useful for the insertion of multiple heterologous sequences. The dCVA can reproductively replicate (with an amplification ratio of greater than 10) in a human cell line (such as human 293, 143B, and MRC-5 cell lines), which then enable the optimization by further mutation useful for a virus-based vaccination strategy (see, e.g., WO 2011/092029).

In a preferred embodiment of the application, the MVA vectors are MVA-BN vectors, such as that described in WO 2018/229711, which is incorporated herein by reference.

According to embodiments of the application, the MVA vector(s) comprise a nucleic acid that encodes one or more HIV immunogens having the amino acid sequences selected from the group consisting of SEQ ID NOs: 1-4. Preferably, the one or more MVA vectors together encode four mosaic HIV immunogens having the amino acid sequences of SEQ ID NO: 1, SEQ ID NO: 2, SEQ ID NO: 3, and SEQ ID NO: 4. A particularly useful but non-limiting example of an MVA vaccine that can be used for the methods described herein is MVA-mBN414, as described in example 7 of WO 2018/229711.

Nucleic acid sequences encoding the mosaic HIV immunogens can be inserted into one or more intergenic regions (IGR) of the MVA. In certain embodiments, the IGR is selected from IGR07/08, IGR 44/45, IGR 64/65, IGR 88/89, IGR 136/137, and IGR 148/149. In certain embodiments, less than 5, 4, 3, or 2 IGRs of the recombinant MVA comprise heterologous nucleotide sequences encoding an HIV immunogen, such as a mosaic HIV immunogen. The heterologous nucleotide sequences can, additionally or alternatively, be inserted into one or more of the naturally occurring deletion sites, in particular into the main deletion sites I, II, III, IV, V, or VI of the MVA genome. In certain embodiments, less than 5, 4, 3, or 2 of the naturally occurring deletion sites of the recombinant MVA comprise heterologous nucleotide sequences encoding mosaic HIV immunogens.

The number of insertion sites of MVA comprising heterologous nucleotide sequences encoding HIV immunogens can be 1, 2, 3, 4, 5, or more. In certain embodiments, the heterologous nucleotide sequences are inserted into 4, 3, 2, or fewer insertion sites. Preferably, two insertion sites are used. In certain embodiments, three insertion sites are used. Preferably, the recombinant MVA comprises at least 2, 3, 4, 5, 6, or 7 genes inserted into 2 or 3 insertion sites.

The recombinant MVA viruses provided herein can be generated by routine methods known in the art. Methods to obtain recombinant poxviruses or to insert exogenous coding sequences into a poxviral genome are well known to the person skilled in the art. For example, methods for standard molecular biology techniques such as cloning of DNA, DNA and RNA isolation, Western blot analysis, RT-PCR, PCR amplification techniques, techniques for the handling and manipulation of viruses, and techniques and know how for the handling, manipulation and genetic engineering of MVA are described in standard textbooks and manuals.

For the generation of the various recombinant MVAs disclosed herein, different methods can be applicable. The DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences.

According to a preferred embodiment, a cell of a suitable cell culture such as, e.g., CEF cells, can be infected with a poxvirus. The infected cell can be, subsequently, transfected with a first plasmid vector comprising a foreign or heterologous gene or genes, preferably under the transcriptional control of a poxvirus expression control element. As explained above, the plasmid vector also comprises sequences capable of directing the insertion of the exogenous sequence into a selected part of the poxviral genome. Optionally, the plasmid vector also contains a cassette comprising a marker and/or selection gene operably linked to a poxviral promoter.

Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In case, this gene shall be introduced into a different insertion site of the poxviral genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can at first be transfected by the plasmid vector comprising the foreign gene and, then, infected with the poxvirus. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E. coli or another bacterial species between a vaccinia virus genome cloned as a bacterial artificial chromosome (BAC) and a linear foreign sequence flanked with DNA sequences homologous to sequences flanking the desired site of integration in the vaccinia virus genome

The heterologous nucleic acid encoding one or more mosaic HIV immunogens can be under the control of (i.e., operably linked to) one or more poxvirus promoters. In certain embodiments, the poxvirus promoter is a Pr7.5 promoter, a hybrid early/late promoter, or a PrS promoter, a PrS5E promoter, a synthetic or natural early or late promoter, or a cowpox virus ATI promoter.

In certain embodiments of the application, an MVA vector useful for methods of the application expresses four HIV immunogens respectively having the amino acid sequences of SEQ ID NOs: 1, 2, 3, and 4.

### Immunogenic Compositions

Immunogenic compositions are compositions comprising an immunogenically effective amount of a purified or partially purified adenovirus (e.g. Ad26) or poxvirus (e.g. MVA) vector for use in methods of the application. The adenovirus and poxvirus vectors can encode any HIV immunogens in view of the present disclosure, and preferably encode one or more HIV immunogens selected from the group consisting of SEQ ID NOs: 1-4. The one or more HIV immunogens encoded by the adenovirus (e.g. Ad26) vector can be different from, but preferably are the same as the one or more HIV immunogens encoded by the poxvirus (e.g. MVA) vector. Immunogenic compositions can be formulated as a vaccine, according to methods well known in the art. Such compositions can include adjuvants to enhance immune responses. The optimal ratios of each component in the formulation can be determined by techniques well known to those skilled in the art in view of the present disclosure.

As used herein, “an immunogenically effective amount” or “immunologically effective amount” means an amount of a composition or vector sufficient to induce a desired immune effect or immune response in a subject in need thereof. In one embodiment, an immunogenically effective amount means an amount sufficient to induce an immune response in a subject in need thereof, preferably a safe and effective immune response in a human subject in need thereof. In another embodiment, an immunogenically effective amount means an amount sufficient to produce immunity in a subject in need thereof, e.g., provide a therapeutic effect against a disease such as HIV infection. An immunogenically effective amount can vary depending upon a variety of factors, such as the physical condition of the subject, age, weight, health, etc. An immunogenically effective amount can readily be determined by one of ordinary skill in the art in view of the present disclosure.

An immunogenically effective amount can be administered in a single step (such as a single injection), or multiple steps (such as multiple injections), or in a single composition or multiple compositions. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

As general guidance, an immunogenically effective amount when used with reference to a recombinant viral vector can range from about 10⁶ viral particles (vps), plaque forming units (pfus) or infectious units (IU) to about 10¹² viral particles or infectious units, for example 10⁶, 10⁷, 10⁸, 10⁹, 10¹⁰, 10¹¹, or 10¹² viral particles or infectious units.

In one embodiment, an immunogenic composition is an adenovirus (preferably Ad26) vaccine used for initial administration to induce an immune response. According to embodiments of the application, an adenovirus (preferably Ad26) vaccine comprises an immunogenically effective amount of one or more adenovirus (preferably Ad26) vectors together encoding four HIV immunogens having the amino acid sequences of SEQ ID NO: 1, SEQ NO: 2, SEQ ID NO: 3 and SEQ ID NO: 4, and a pharmaceutically acceptable carrier. The HIV immunogens can be encoded by the same adenovirus (preferably Ad26) vector or different adenovirus (preferably Ad26) vector, such as one, two, three, four or more adenovirus (preferably Ad26) vectors.

The immunogenically effective amount of the one or more adenovirus (preferably Ad26) vectors can be about 10⁸, 10⁹, 10¹⁰, 10¹¹, or 10¹² viral particles (vps), preferably about 10⁹ to 10¹¹ viral particles, and more preferably about 10¹⁰ viral particles, such as for instance about 0.5×10¹⁰, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 10×10¹⁰ viral particles. In certain embodiments, the immunogenically effective amount is about 5×10⁹ to about 1×10¹¹ viral particles, preferably about 5×10¹⁰ viral particles, such that the one or more adenovirus (preferably Ad26) vectors are administered at a total dose of about 5×10⁹ to about 1×10¹¹ viral particles per immunization step.

The immunogenically effective amount can be from one adenovirus (preferably Ad26) vector or multiple adenovirus (preferably Ad26) vectors. For example, a total administered dose of about 5×10⁹ to about 1×10¹¹ viral particles, such as for instance about 5×10¹⁰ viral particles, in the adenovirus (preferably Ad26) vaccine can be from four adenovirus (preferably Ad26) vectors each encoding a different mosaic HIV immunogen, such as those shown in SEQ ID NOs: 1, 2, 3, and 4.

In a particular embodiment, the immunogenically effective amount of Ad26 vectors together encoding SEQ ID NOs: 1, 2, 3, and 4 consists of four adenovirus vectors, namely a first Ad26 vector encoding the HIV immunogen of SEQ ID NO: 1, a second Ad26 vector encoding the HIV immunogen of SEQ ID NO: 2, a third Ad26 vector encoding the HIV immunogen of SEQ ID NO: 3, and a fourth Ad26 vector encoding the HIV immunogen of SEQ ID NO: 4.

In such embodiments where an adenovirus (preferably Ad26) vaccine comprises more than one adenovirus (preferably Ad26) vector, the adenovirus (preferably Ad26) vectors can be included in the composition in any ratio to achieve the desired immunogenically effective amount. Preferably, when the immunogenically effective amount of the adenovirus (preferably Ad26) vectors consists of four adenovirus (preferably Ad26) vectors, the first, second, third, and fourth adenovirus (preferably Ad26) vectors are administered at a 1:1:1:1 ratio of viral particles (vps).

A poxvirus (preferably an MVA) vaccine that is useful in methods of the application comprises an immunogenically effective amount of one or more poxvirus (preferably MVA) vectors together encoding the four HIV immunogens having the amino acid sequences of SEQ ID NO: 1, SEQ ID NO: 2, SEQ ID NO: 3, SEQ ID NO: 4, and a pharmaceutically acceptable carrier. The HIV immunogens expressed by poxvirus (preferably MVA) vectors can be encoded by a single poxvirus (preferably MVA) vector, or multiple poxvirus (preferably MVA) vectors, such as one, two, or more poxvirus (preferably MVA) vectors. In certain advantageous embodiments, the HIV immunogens are expressed by a single MVA vector.

The immunogenically effective amount of the one or more poxvirus (preferably MVA) vectors in the poxvirus (preferably MVA) vaccine can be about 10⁶, 10⁷, 10⁸, 10⁹, or 10¹⁰ infectious units (IU), preferably about 10⁷ to 10⁹ IU, and more preferably about 2×10⁸ IU, such as for instance about 0.5×10⁸, 1×10⁸, 2×10⁸, 3×10⁸, 4×10⁸, or 5×10⁸ IU. In certain embodiments, the immunogenically effective amount is about 1×10⁷ to about 5×10⁸ IU, preferably about 2×10⁸ IU, such that the one or more poxvirus (preferably MVA) vectors are administered at a total dose of about 1×10⁷ to about 5×10⁸ IU, preferably about 2×10⁸ IU per immunization step.

The immunogenically effective amount can be from one poxvirus (preferably MVA) vector or multiple poxvirus (preferably MVA) vectors. For example, in some embodiments, a total administered dose of about 1×10⁷ to about 5×10⁸ IU, such as for instance about 1×10⁷, 5×10⁷, 1×10⁸, 2×10⁸, 5×10⁸ IU, or any dose in between, in the poxvirus (preferably MVA) vaccine can be from two poxvirus (preferably MVA) vectors together encoding four HIV immunogens having the amino acid sequences of SEQ ID NO: 1, SEQ ID NO: 2, SEQ ID NO: 3, and SEQ ID NO: 4, for example a first MVA vector encoding the HIV immunogens of SEQ ID NOs: 1 and 3, and a second MVA vector encoding the HIV immunogens of SEQ ID NO: 2 and SEQ ID NO: 4, wherein preferably the first and second MVA vectors are administered at a 1:1 ratio of IU. In more preferred embodiments, a total administered dose of about 1×10⁷ to about 5×10⁸ IU, such as for instance about 1×10⁷, 5×10⁷, 1×10⁸, 2×10⁸, 5×10⁸ IU, or any dose in between, in the poxvirus (preferably MVA) vaccine can be from a single poxvirus (preferably MVA) vector encoding four HIV immunogens having the amino acid sequences of SEQ ID NOs: 1, 2, 3, and 4.

The preparation and use of immunogenic compositions are well known to those of ordinary skill in the art. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can also be included. The immunogenic compositions used in methods of the application, can be formulated for administration according to any method known in the art in view of the present disclosure, and are preferably formulated for intramuscular administration.

The vaccine compositions of the application can comprise other immunogens. The other immunogens used in combination with the adenovirus (preferably Ad26) and/or poxvirus (preferably MVA) vectors can be, for example, other HIV immunogens and nucleic acids expressing them.

The immunogenic compositions useful in the application can further optionally comprise adjuvants. Adjuvants suitable for co-administration in accordance with the vaccines of the application should be ones that are potentially safe, well tolerated and effective in people. Non-limiting examples include QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Aluminium salts such as Aluminium Phosphate (e.g. AdjuPhos) or Aluminium Hydroxide, and MF59.

The immunogenic compositions used for generating an immune response according to embodiments of the application comprise a pharmaceutically acceptable carrier, such as a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., intramuscular, subcutaneous, oral, intradermal, cutaneous, intramucosal (e.g., gut), intranasal or intraperitoneal routes. Preferably, the pharmaceutically acceptable carrier included in the compositions of the application is suitable for intramuscular administration.

### Broadly Neutralizing Antibodies

Broadly neutralizing antibodies in combination with vaccine regimens have the potential to reduce or eliminate reactivated infected cells via Fc-mediated innate effector functions and to expand vaccine-induced T-cell responses either by slowing viral rebound, and thus allowing controlled virus-induced expansion of T-cell responses, or alternatively by forming immune complexes and augmenting T-cell responses by a vaccinal effect.

The term “antibody” or “immunoglobulin (Ig)” is used in the broadest sense and includes monoclonal antibodies, polyclonal antibodies, chimeric antibodies, multivalent antibodies, multispecific antibodies, and antibody fragments. The monoclonal antibodies can be full-length or intact monoclonal antibodies, or antigen binding fragments thereof. Examples of the multispecific antibodies include bispecific antibodies or trispecific antibodies. An antibody typically comprises both light chains and heavy chains. The light chains of antibodies (immunoglobulins) from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., lgG1, lgG2, lgG3, lgG4, lgA1, and lgA2. The heavy chain constant domains that correspond to the different classes of immunoglobulins are called α, δ, ε, γ, and μ, respectively. The subunit structures and three-dimensional configurations of different classes of immunoglobulins are well known.

As used herein, the term “anti-HIV broadly neutralizing antibody” or “anti-HIV bNAb” refers to an antibody that recognizes and neutralizes multiple HIV viral strains. For example, an anti-HIV bNAb can recognize a conserved region of an HIV antigen (e.g., gp120 of HIV) and inhibits the effect(s) of the recognized antigen in at least 2, 3, 4, 5, 6, 7, 8, 9 or more different strains of HIV, the strains belonging to the same or different clades. In some embodiments, a broadly neutralizing antibody can neutralize multiple HIV species belonging to at least 2, 3, 4, 5, or 6 different clades.

In accordance with embodiments of the application, two or more anti-HIV bNAbs can be used for methods of the application. In certain embodiments two, three, four, five, six, seven, eight, nine, ten, or more anti-HIV bNAbs can be used for methods of the application. Including more anti-HIV bNAbs ensures broader coverage of HIV variants and decreases the chance for escape mutants. However, each antibody will need to be manufactured and controlled as a pharmaceutical product and thus including more anti-HIV bNAbs will add to costs and complexity of the method of the invention. In preferred embodiments a balance is found by using two, three, or four anti-HIV bNAbs for methods of the application. Most preferably three anti-HIV bNAbs are used for methods of the invention. The two or more anti-bNAbs can be administered together in the same composition or administered in two or more different compositions. In certain embodiments, the two or more anti-HIV bNAbs are encompassed in a single multimeric antibody binding two or more different epitopes. Such multimeric anti-HIV bNAbs have been described (see e.g. Wagh K, et al. (2018) PLoS Pathog 14(3): e1006860), and the skilled person thus is aware of such antibodies and can obtain such antibodies; non-limiting examples are 10E8v2.0-iMAb, which targets membrane proximal external region (MPER) on Env and host-cell CD4 (described in Huang Y, et al., Cell. 2016; 165(7):1621-31), and 3BNC117-PGT135, which targets CD4 binding site and V3 glycan epitopes on Env (described in Bournazos S, et al., Cell. 2016; 165(7):1609-20).

As used herein, “epitope” refers to a portion of an antigen to which an antibody specifically binds. Epitopes usually consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

In accordance with embodiments of the application, each individually of the two or more anti-HIV bNAbs useful for methods of the application bind to an epitope or region of HIV gp120 envelope selected from the group consisting of: (i) CD4-binding site (CD4bs) (ii) third variable loop (V3) and/or high mannose patch comprising a N332 oligomannose glycan; (iii) second variable loop (V2) and/or Env trimer apex; (iv) gp120/gp41 interface; or (v) silent face of gp120.

Exemplary anti-HIV bNAbs include, but are not limited to, those described in WO2012/158948; WO2015/048770; WO 2012/030904; WO 2013/055908; WO2013/086533; WO2017/106346; WO2019/226829; US 2012/0288502; US 2015/0361160; Walker et al. Nature. 477: 466-470, 2011; Mouquet et al. Proc. Natl. Acad. Sci. 109(47): E3268-E3277, 2012; Julien et al., PLoS Pathog. 9: e1003342, 2013; Kong et al., Nat. Struc. Mol. Biol. 20: 796-803, 2013; Scheid et al., Nature. 458: 636-640, 2009; and Roben et al., J Virol. 68: 4821-4828, 1994; the disclosures of which are incorporated herein by reference in entirety.

In some embodiments, the two or more anti-HIV bNAbs useful for the invention are selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01, M2, CH103, 1NC9, 12A12, VRC01, VRC01-LS, VRC07-523, VRC07-523LS, N6, 3BNC117, NIH45-46, PGV04 (VRC-PG04), GS-9722, GS-9722, PGT-121.60, PGT-121.66, PGT-121, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03.

In certain embodiments, the two or more anti-HIV bNAbs are selected from the group consisting of VRC01, 3BNC117, VRC01-LS, VRC07-523LS, 10-1074, PGT121, and PGDM1400, preferably the two or more anti-HIV bNAbs are selected from the group consisting of PGT121, PGDM1400, and VRC07-523LS. In a preferred embodiment the bNAbs consist of PGT121, PGDM1400, and VRC07-523LS. PGT-121 is described in (Walker L M, et al. Nature. 2011; 477(7365):466-70) and in U.S. Pat. No. 10,836,811, the entire contents of each of which are incorporated herein by reference. PGDM1400 is described in (Sok D, et al. Proc Natl Acad Sci USA. 2014; 111(49):17624-9) and in U.S. Pat. No. 10,093,720 and in WO 2020/106713, the entire contents of each of which are incorporated herein by reference. VRC07-523LS is described in (Rudicell R S, et al. J Virol. 2014; 88(21):12669-82) and in U.S. Pat. No. 9,695,230 and WO 2017/079479, the entire contents of each of which is incorporated herein by reference.

In certain embodiments, the two or more anti-HIV bNAbs are selected from the group consisting of an antibody having variable regions that comprise amino acid sequences that are at least 90% identical, preferably at least 91%, 92%, 93%, 94%, 95%, 96%, 97%, 98%, 99%, or 100% identical, to SEQ ID NOs: 9 and 10; an antibody having variable regions that comprise amino acid sequences that are at least 90% identical, preferably at least 91%, 92%, 93%, 94%, 95%, 96%, 97%, 98%, 99%, or 100% identical, to SEQ ID NOs: 13 and 14; and an antibody having variable regions that comprise amino acid sequences that are at least 90% identical, preferably at least 91%, 92%, 93%, 94%, 95%, 96%, 97%, 98%, 99%, or 100% identical, to SEQ ID NOs: 17 and 18.

In certain embodiments, the two or more anti-HIV bNAbs are selected from the group consisting of an antibody having a heavy chain and light chain that comprise amino acid sequences that are at least 90% identical, preferably at least 91%, 92%, 93%, 94%, 95%, 96%, 97%, 98%, 99%, or 100% identical, to SEQ ID NOs: 11 and 12, respectively; an antibody having a heavy chain and light chain that comprise amino acid sequences that are at least 90% identical, preferably at least 91%, 92%, 93%, 94%, 95%, 96%, 97%, 98%, 99%, or 100% identical, to SEQ ID NOs: 15 and 16, respectively; and an antibody having a heavy chain and light chain that comprise amino acid sequences that are at least 90% identical, preferably at least 91%, 92%, 93%, 94%, 95%, 96%, 97%, 98%, 99%, or 100% identical, to SEQ ID NOs: 19 and 20, respectively.

In certain embodiments, PGT121 comprises a heavy chain and a light chain that comprise amino acid sequences set forth in SEQ ID NOs: 11 and 12, respectively. In certain embodiments, PGDM1400 comprises a heavy chain and a light chain that comprise amino acid sequences set forth in SEQ ID NOs: 15 and 16, respectively. In certain embodiments, VRC07-523LS comprises a heavy chain and a light chain that comprise amino acid sequences set forth in SEQ ID NOs: 19 and 20, respectively.

In certain embodiments, the two or more anti-HIV bNAbs compete with the bNAbs described herein for binding to multiple HIV strains, or have identical complementarity-determining regions (CDRs) as those of the bNAbs described herein. In certain embodiments, variant anti-HIV bNAbs can be used in the methods of the invention, which variant anti-HIV bNAbs comprise variable regions that comprise amino acid sequences that are at least 90% identical, preferably at least 91%, 92%, 93%, 94%, 95%, 96%, 97%, 98%, 99% identical to the variable regions of the antibodies described herein, and that bind to the same epitope as the antibodies described herein. Non-limiting examples of variants of PGDM1400 have been described in WO2020/106713, incorporated herein by reference. Non-limiting examples of variants of PGT121 have been described in WO2019/226829 and WO2017/106346, each of which is incorporated herein by reference. Non-limiting examples of variants of VRC07-523, including VRC07-523LS, as well as an exemplary constant region of an IgG1 light chain and of an IgG1 heavy chain with the LS mutation, have been described in US 2021/0079070, incorporated herein by reference.

In certain embodiments, the two or more anti-HIV bNAbs include one or more variants of the anti-HIV bNAbs described herein. As is known to the skilled person, antibodies can be engineered, for example to decrease or increase affinity, decrease or extend in vivo half-life, decrease or increase Fc-mediated effector functions, and/or to reduce side-effects, etc. Hence, variants of antibodies that bind to the same target epitope as known antibodies can be obtained by the skilled person using known methods, and such variants are within the scope of the HIV bNAbs described herein or elsewhere. In certain embodiments, such variants include variants with higher affinity, e.g., by a limited number (e.g., 1, 2, or 3) of mutations in one or more of the complementarity determining regions. In certain embodiments, such variants include variants with increased in vivo half-life, e.g., by a limited number (e.g., 1, 2, 3, 4, or 5) of mutations in the constant region of the heavy chain. In certain non-limiting embodiments, to optimize in vivo half-life, an LS mutation can be added to the FcRn receptor binding region of an antibody. This mutation has no effect on neutralization breadth or potency but increases the half-life of the antibody by 2- to 3-fold in both humanized mice and non-human primates. Thus, an anti-HIV bNAb comprising an LS mutation can be administered fewer times than an antibody without the LS mutation. Addition of the LS mutation to a monoclonal antibody is known to the person of skill in the art (see, U.S. Pat. No. 8,394,925, the entire content of which is incorporated herein by reference).

The anti-bNAbs provided herein can be generated by routine methods known in the art in view of the present disclosure. Anti-HIV bNAbs useful for methods of the application can be prepared using methods known in the art, such as those described in WO2012/158948, WO2015/048770, WO 2012/030904, WO 2013/055908, US 2012/0288502, etc., the relevant disclosures of which are incorporated herein by reference.

The formulation of and delivery methods of pharmaceutical compositions will generally be adapted according to the site and the disease to be treated. Exemplary formulations include, but are not limited to, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. The bNAbs used in methods of the application, can be formulated for administration according to any method known in the art in view of the present disclosure, and are preferably formulated for intravenous administration.

The dose of two or more anti-HIV bNAbs depends at least on toxicity, the method of delivery, and the pharmaceutical formulation, and can be determined by the clinician using conventional dose escalation studies.

In certain embodiments, the two or more anti-HIV bNAbs are administered once or twice.

The dose of each anti-HIV bNAbs can be from about 5 mg/kg to about 40 mg/kg, such as for instance about 5 mg/kg, 15 mg/kg, 20 mg/kg, 25 mg/kg, 30 mg/kg, 35 mg/kg, 40 mg/kg, or any dose in between, per administration. In certain embodiments, each anti-HIV bNAb is administered intravenously at a dose of about 5-40 mg/kg of the anti-HIV bNAb, preferably about 10-20 mg/kg of each anti-HIV bNAb, per administration.

### Method of Inducing an Immune Response Against HIV Infection

The adenovirus and poxvirus vaccine compositions described above can be used in the methods of the application described herein. The methods of the application relate to inducing an immune response against a human immunodeficiency virus (HIV) in an HIV-infected subject undergoing antiretroviral therapy. The methods of administering adenovirus and poxvirus vaccines according to embodiments of the application are effective to induce an immune response against one or multiple clades of HIV.

The application relates to a method of inducing an immune response against a human immunodeficiency virus (HIV) in an HIV-infected human subject undergoing antiretroviral therapy (ART), the method comprising:

(i) administering to the human subject an adenovirus vaccine comprising one or more adenovirus vectors together encoding HIV gag, pol, and env immunogens, preferably four HIV immunogens having the amino acid sequences of SEQ ID NO: 1, SEQ ID NO: 2, SEQ ID NO: 3, and SEQ ID NO: 4, and a pharmaceutically acceptable carrier;

(ii) administering to the human subject a poxvirus vaccine comprising one or more poxvirus vectors together encoding the HIV gag, pol, and env immunogens, preferably, the four HIV immunogens and a pharmaceutically acceptable carrier; and

(iii) administering to the human subject two or more anti-HIV broadly neutralizing antibodies (bNAbs) and one or more pharmaceutically acceptable carriers.

In certain embodiments, the one or more adenovirus vectors are adenovirus 26 (Ad26) vectors. In certain embodiments, the adenovirus vaccine comprises a first Ad26 vector encoding the HIV immunogen of SEQ ID NO: 1, a second Ad26 vector encoding the HIV immunogen of SEQ ID NO: 2, a third Ad26 vector encoding the HIV immunogen of SEQ ID NO: 3, and a fourth Ad26 vector encoding the HIV immunogen of SEQ ID NO: 4.

In certain embodiments, the one or more poxvirus vectors are Modified Vaccinia Ankara (MVA) vectors. In certain embodiments, the poxvirus vaccine consists of a single MVA vector encoding the four HIV immunogens.

In certain embodiments, the two or more anti-HIV bNAbs bind to an epitope or region of HIV gp120 envelope selected from the group consisting of (i) CD4-binding site (CD4bs) (ii) third variable loop (V3) and/or high mannose patch comprising a N332 oligomannose glycan; (iii) second variable loop (V2) and/or Env trimer apex; (iv) gp120/gp41 interface; or (v) silent face of gp120.

In certain embodiments, the two or more anti-HIV bNAbs are selected from the group consisting of VRC01, 3BNC117, VRC01-LS, VRC07-523LS, 10-1074, PGT121, and PGDM1400, preferably the two or more anti-HIV bNAbs are selected from the group consisting of PGT121, PGDM1400, and VRC07-523LS.

In certain embodiments, the one or more adenovirus vectors together are administered at a total dose of about 5×10⁹ to about 1×10¹¹ viral particles (vp), preferably about 5×10¹⁰ vp, of the one or more adenovirus vectors, per administration. In preferred embodiments of the methods of the invention, the one or more adenovirus vectors are administered only once.

In certain embodiments, the one or more poxvirus vectors together are administered at a total dose of about 1×10⁷ to about 5×10⁸ infectious units (IU), preferably about 2×10⁸ IU, of the one or more poxvirus vectors, per administration.

In certain embodiments, the poxvirus vaccine is administered 8-14 weeks, such as 8, 9, 10, 11, 12, 13 or 14 weeks, preferably 12 weeks, after the adenovirus vaccine is initially administered. In preferred embodiments of the methods of the invention, the one or more poxvirus vectors are administered only once.

In certain embodiments, each anti-HIV bNAb is administered at a dose of about 5-40 mg/kg of the anti-HIV bNAb, preferably about 10-20 mg/kg of each anti-HIV bNAb, per administration.

In certain embodiments, the anti-HIV bNAb is administered 20-30 weeks, such as 20, 21, 22, 23, 24, 25, 26, 27, 28, 29 or 30 weeks, preferably 24 weeks, after the adenovirus vaccine is initially administered.

In certain embodiments, one or more of the anti-HIV bNAbs is administered one or two times at about 20-30 weeks, such as 20, 21, 22, 23, 24, 25, 26, 27, 28, 29 or 30 weeks, preferably at about 24 and about 28 weeks, after the adenovirus vaccine is initially administered. In certain embodiments of the methods of the invention, the two or more anti-HIV bNAbs are administered once or twice. The method described herein does not require chronic bNAb administration, but rather a limited number of administrations.

In certain embodiments, a method of inducing an immune response against a human immunodeficiency virus (HIV) in an HIV-infected human subject undergoing antiretroviral therapy (ART), the method comprises:

(i) intramuscularly administering to the human subject an adenovirus 26 (Ad26) vaccine comprising one or more Ad26 vectors together encoding four HIV immunogens having the amino acid sequences of SEQ ID NO: 1, SEQ ID NO: 2, SEQ ID NO: 3, and SEQ ID NO: 4, and a pharmaceutically acceptable carrier, in a total dose of about 5×10⁹ to about 1×10¹¹ viral particles (vp), preferably about 5×10¹⁰ vp, of the Ad26 vectors;

(ii) intramuscularly administering to the human subject an Modified Vaccinia Ankara (MVA) vaccine comprising one or more MVA vectors, preferably one or more MVA-BN vectors, encoding the four HIV immunogens and a pharmaceutically acceptable carrier, in a total dose of about 1×10⁷ to about 5×10⁸ infectious units (IU), preferably about 2×10⁸ IU, of the one or more MVA vectors, wherein the MVA vaccine, is administered 8-14 weeks, preferably 12 weeks, after the Ad26 vaccine is initially administered in step (i); and

(iii) intravenously administering to the human subject PGT121, PGDM1400, and VRC07-523LS anti-HIV broadly neutralizing antibodies (bNAbs) and a pharmaceutically acceptable carrier, at a dose of about 5 mg/kg to about 40 mg/kg of each anti-HIV bNAb, per administration, preferably the anti-HIV bNAbs PGT121 and PGDM1400 are administered one or two times at 20-30 weeks after the Ad26 vaccine is initially administered in step (i); more preferably, at a dose of 10-20 mg/kg of each anti-HIV bNAb per administration at 24 and 28 weeks after the Ad26 vaccine is initially administered in step (i).

In certain embodiments, PGT121 is administered at a dose of 20 mg/kg of PGT121, per administration, at 24 and 28 weeks after the Ad26 vaccine is initially administered in step (i).

In certain embodiments, PGDM1400 is administered at a dose of 20 mg/kg of PGDM1400, per administration, at 24 and 28 weeks after the Ad26 vaccine is initially administered in step (i).

In certain embodiments, VRC07-523LS is administered at a dose of 10 mg/kg of VRC07-523LS at 24 weeks after the Ad26 vaccine is initially administered in step (i).

Any of the vaccine compositions described herein can be used in a method according to the application. Embodiments of the Ad26 vaccine; MVA vaccine; Ad26 vectors; MVA vectors; HIV immunogens encoded by the Ad26 and MVA vectors, etc. that can be used in the methods of the application are discussed in detail above and in the illustrative examples below.

According to embodiments of the application, “inducing an immune response” when used with reference to the methods described herein encompasses causing a desired immune response or effect in a subject in need thereof against an HIV infection, preferably for therapeutic purposes. “Inducing an immune response” also encompasses providing a therapeutic immunity for treating against a pathogenic agent, i.e., HIV. As used herein, the term “therapeutic immunity” or “therapeutic immune response” means that the HIV-infected vaccinated subject is able to control an infection with the pathogenic agent, i.e., HIV, against which the vaccination was done. In one embodiment, “inducing an immune response” means producing an immunity in a subject in need thereof, e.g., to provide a therapeutic effect against a disease such as HIV infection. In certain embodiments, “inducing an immune response” refers to causing or improving cellular immunity, e.g., T cell response, against HIV. In certain embodiments, “inducing an immune response” refers to causing or improving a humoral immune response against HIV. In certain embodiments, “inducing an immune response” refers to causing or improving a cellular and a humoral immune response against HIV. Typically, the administration of the Ad26 and MVA vaccine compositions according to embodiments of the application will have a therapeutic aim to generate an immune response against HIV after HIV infection or development of symptoms characteristic of HIV infection. In certain embodiments, the induced immune response in the subject in which ART has successfully suppressed replication of HIV in the blood stream is such that the subject can discontinue the ART and still maintains control of viral replication in the blood stream for at least 8, preferably at least 12, 16, 20, or 24 weeks after discontinuation of the ART, more preferably for at least 30, 40, 50, 60, or 70 or more weeks after discontinuation of the ART.

The patient population for treatment according to the methods of the application described herein is HIV-infected human subjects, particularly HIV-infected human subjects undergoing antiretroviral therapy (ART). The terms “HIV infection” and “HIV-infected” as used herein refer to invasion of a human host by HIV. As used herein, “an HIV-infected human subject” refers to a human subject in whom HIV has invaded and subsequently replicated and propagated within the human host, thus causing the human host to be infected with HIV or have an HIV infection or symptoms thereof. An “HIV-infected human subject” has been diagnosed with HIV infection, i.e., tests positive in a screen for HIV infection, e.g. using any assay that is US FDA-approved.

As used herein, “undergoing antiretroviral therapy” refers to a human subject, particularly an HIV-infected human subject, that is being administered, or who has initiated treatment with antiretroviral drugs. According to embodiments of the application, the antiretroviral therapy (ART) is started prior to the first administration of the adenovirus (e.g. Ad26) vaccine, for instance, about 2 to 6 weeks prior, such as about 2, 3, 4, 5, or 6 weeks prior, or 2-48 months prior, such as about 2, 3, 5, 6, 8, 12, 16, 20, 24, 30, 36, 42, or 48 months prior, or longer. In certain embodiments the ART is started at least about 44-52 weeks, preferably at least about 48 weeks prior to the first administration of the adenovirus (e.g. Ad26) vaccine. In a subject undergoing antiretroviral therapy, the antiretroviral therapy is continued during administration of the vaccine part (i.e. adenovirus and poxvirus vectors) of the regimen of the application. ART is considered “suppressive” as used herein if the subject has plasma HIV RNA levels at less than 50 copies/mL for a certain period of time, including the possibility of blips. The term “stable suppressive” ART as used herein means that the suppressive ART regimen is not modified for a certain period of time.

In certain embodiments, a human subject undergoing antiretroviral therapy is on current stable suppressive ART for at least 48 weeks, meaning that while receiving the same ART regimen the subject has plasma HIV ribonucleic acid (RNA) levels at less than 50 copies/mL for at least 48 weeks prior to initiation of a regimen according to the application. However, the human subject can have one or more blips (i.e., instances) of plasma HIV RNA greater than 50 copies/ml to less than 200 copies/ml within this period, such as within the 12-week period prior to the initiation of the regimen, provided that screening immediately prior to initiation of the regimen is less than 50 copies/ml.

A subject undergoing ART can be administered or treated with any antiretroviral drugs known in the art in view of the present disclosure. ART are medications that treat HIV, although the drugs do not kill the virus or remove the virus from the body. However, when taken in combination they can prevent the growth of the virus. When the virus is slowed down, so is HIV disease. Antiretroviral drugs are referred to as ARV. Combination ARV therapy (cART) is referred to as highly active ART (HAART). Typically, an ART regimen includes at least three antiviral compounds, e.g., two different reverse transcriptase inhibitors plus either a non-nucleoside reverse transcriptase inhibitor or protease inhibitor or integrase inhibitor.

One of ordinary skill in the art will be able to determine the appropriate antiretroviral treatment, frequency of administration, dosage of the ART, etc. so as to be compatible with simultaneous administration of the regimens of the application. Examples of antiretroviral drugs used for ART include, but are not limited to nucleoside reverse transcriptase inhibitors (NRTIs, non-limiting examples of which include zidovudine, didanosine, stavudine, lamivudine, abacavir, tenofovir, combivir [combination of zidovudine and lamivudine], trizivir [combination of zidovudine, lamivudine and abacavir], emtricitabine, truvada [combination of emtricitabine and tenofovir], and epzicom [combination of abacavir and lamivudine]), non-nucleoside reverse transcriptase inhibitors (NNRTIs, non-limiting examples of which include nevirapine, delavirdine, efavirenz, etravirine, and rilpivirine), protease inhibitors (PIs, non-limiting examples of which include saquinavir, indinavir, ritonavir, nelfinavir, amprenavir, lopinavir/ritonavir, atazanavir, fosamprenavir, tipranavir, darunavir), integrase inhibitors (INSTIs, non-limiting examples including raltegravir, elvitegravir, and dolutegravir), and fusion inhibitors, entry inhibitors and/or chemokine receptor antagonists (FIs, CCR5 antagonists; non-limiting examples including enfuvirtide, maraviroc, vicriviroc, and cenicriviroc).

In certain embodiments, the human subject continues undergoing suppressive ART during treatment.

According to embodiments of the application, a poxvirus vaccine is administered after an adenovirus vaccine is initially administered. In certain embodiments, the poxvirus vaccine is administered 8-14 weeks, such as 8, 10, 12, or 14 weeks, after the adenovirus vaccine is initially administered.

Further administrations are possible, and embodiments of the disclosed methods also contemplate administration of such additional immunizations with immunogenic compositions containing adenovirus vectors and poxvirus vectors. Any of the adenovirus vectors and poxvirus vectors described herein can be used in additional immunizations. Preferably however, each of the vaccine components (adenovirus and poxvirus) is administered only once.

The vaccine compositions can be administered by any method known in the art in view of the present disclosure, and administration is typically via intramuscular, intradermal or subcutaneous administration, preferably intramuscular administration. Intramuscular administration can be achieved by using a needle to inject a suspension or solution of the adenovirus and/or MVA vectors. An alternative is the use of a needleless injection device to administer the composition (using, e.g., Biojector™) or a freeze-dried powder containing the vaccine.

Other modes of administration, such as intravenous, cutaneous, intradermal, oral, intratracheal, or nasal are also envisaged. For intravenous, cutaneous or subcutaneous injection, the vector will be in the form of a parenterally acceptable aqueous solution which is pyrogen-free and has suitable pH, isotonicity and stability. Those of ordinary skill in the art are well able to prepare suitable solutions using, for example, isotonic vehicles such as Sodium Chloride Injection, Ringer's Injection, and Lactated Ringer's Injection. Preservatives, stabilizers, buffers, antioxidants and/or other additives can be included, as required. A slow-release formulation can also be employed.

Accordingly, another general aspect of the application relates to a method of treating a human immunodeficiency virus (HIV) infection in a human subject in need thereof, comprising:

(i) treating the human subject with an antiretroviral therapy (ART); and

(ii) inducing an immune response against the HIV in the human subject using a method according to an embodiment of the application.

In certain embodiments, the method of treatment further comprises discontinuing the ART treatment of step (i), preferably after an immune response is induced by a method of the application, more preferably after the first administration of the two or more anti-HIV bNAbs. The ART treatment may be resumed if needed at a later stage upon detection of viral rebound.

In certain embodiments, subjects undergo interruption (also referred to as discontinuation, used interchangeably herein) of ART after completion of a regimen according to embodiments of the application. In some embodiments, subjects can undergo antiretroviral analytical treatment interruption (ARV ATI) after completion of a regimen according to embodiments of the application. “Antiretroviral analytical treatment interruption” and “ARV ATI” as used in the application refer to discontinuation of treatment with antiretroviral drugs in order to assess viral suppression and viremic control in the absence of continued ART. Typically, subjects can undergo ARV ATI, i.e., ART can be discontinued, for example when the subject has plasma HIV RNA levels at less than 50 copies/mL for at least about 52 weeks, but a subject can still undergo ARV ATI even if the subject has one or more blips (i.e., instances) of plasma HIV RNA greater than 50 copies/ml to less than 200 copies/ml within this period, provided that the screening immediately prior to ARV ATI shows less than 50 copies/ml of plasma HIV RNA. HIV viral load, e.g., plasma HIV RNA levels, can be measured using known methods in view of the present disclosure, for example, using the Abbott RealTime HIV-1 viral load assay, or the Roche Cobas Taqman HIV-1 viral load assay.

According to embodiments of the application, the ART can be stopped at about 1 to 10 days, such as 1, 2, 3, 4, 5, 6, 7, 8, 9, or 10 days, after the two or more anti-HIV bNAbs are initially administered, preferably 2 days after the two or more anti-HIV bNAbs are initially administered.

Subjects undergoing ARV ATI can be monitored, e.g., by measuring plasma HIV RNA levels. As a non-limiting example, monitoring after the initiation of ARV ATI can occur up to two times per week during the first six weeks when rebound viremia is most likely to occur. “Rebound viremia” is for example defined as plasma HIV RNA levels of greater than 1,000 copies/ml after ARV ATI. ART can be re-initiated in subjects with rebound viremia. Preferably, a subject treated according to the methods of the application will maintain viremic control after ART interruption. As used herein, “maintain viremic control” means “suppress viral replication” which results in levels of virus at such low levels that there is limited weakening of the immune system (which would be the natural course of HIV infection), and maintaining viremic control is in exemplary embodiments defined as at least 24 weeks with plasma HIV RNA of less than 50 copies/mL after ARV ATI. The “maintained viremic control” criterion is in certain exemplary embodiments still deemed to be met if there are one or more instances of plasma HIV RNA greater than 50 copies/ml to less than 1000 copies/ml, as long as the subject does not have plasma HIV RNA levels above 1000 copies/ml on two consecutive determinations at least one week apart.

Typically (not using the methods of the instant invention) human HIV-infected subjects have a return of viremia after 2-3 weeks following ART interruption. Without wishing to be bound by any theories, it is believed that vaccine therapy using an adenovirus vaccine and a poxvirus vaccine according to embodiments of the invention among individuals with fully suppressed HIV will result in a measurable immune response and maintain viremic control after ARV ATI. In some embodiments, subjects can discontinue ART after being treated according to a method of the invention. Discontinuation of ART can be for long periods of time (e.g., at least 24 weeks, preferably longer, e.g., at least about 28, 32, 36, 40, 44, 48, 52 weeks, 16 months, 18, 20, 22, 24 months, or even longer). Such periods of time in which ART is stopped or discontinued are referred to as a “holiday” or “ART holiday” or “treatment holiday”. In other embodiments, vaccine therapy according to the methods of the invention can provide HIV remission, meaning that viral suppression is maintained in the absence of ART. In certain embodiments of the invention, a human subject that received the priming and boosting vaccines of the invention, discontinues ART and maintains viral suppression for at least 24 weeks after discontinuing ART.

In one exemplary regimen of the application, an Ad26 vaccine comprising one or more adenovirus 26 vectors is administered (e.g., intramuscularly) in an amount of about 100 μl to about 2 ml, preferably about 0.5 ml, of a solution containing concentrations of about 10⁸ to 10¹² virus particles/ml. The initial Ad26 vaccination is followed by an MVA vaccine comprising one more MVA vectors administered (e.g., intramuscularly) in an amount of about 100 μl to about 2 ml, preferably about 0.5 ml, of a solution containing concentrations of about 10⁶ to 10⁹ pfu/ml. After administration of the MVA vaccine, two or more anti-HIV bNAbs are administered, preferably followed by a further administration of one or more anti-HIV bNAbs.

The skilled artisan (e.g., practitioner) will appreciate that certain factors may influence the dosage required to effectively treat a subject, including but not limited to the severity of the disease or disorder, previous treatments, the general health and/or age of the subject, and other diseases present.

The invention also relates to a vaccine and two or more, preferably three, bNAbs combination for use in inducing an immune response against a human immunodeficiency virus (HIV) in an HIV-infected human subject undergoing antiretroviral therapy (ART), wherein the vaccine and two or more anti-HIV bNAbs combination comprises an adenovirus, e.g. Ad26, vaccine and a poxvirus, e.g. MVA, vaccine and two or more anti-HIV bNAbs according to embodiments of the invention. The invention yet further relates to use of a vaccine and two or more anti-HIV bNAbs combination in the manufacture of a medicament for inducing an immune response against a human immunodeficiency virus (HIV) in an HIV-infected human subject undergoing antiretroviral therapy (ART), wherein the vaccine and two or more anti-HIV bNAbs combination comprises an adenovirus, e.g. Ad26, vaccine and a poxvirus, e.g. MVA, vaccine and two or more bNAbs according to embodiments of the invention. In another aspect, the invention provides an Ad26 vaccine comprising one or more adenovirus 26 (Ad26) vectors together encoding four HIV immunogens having the amino acid sequences of SEQ ID NO: 1, SEQ ID NO: 2, SEQ ID NO: 3, and SEQ ID NO: 4, and a pharmaceutically acceptable carrier for use in inducing an immune response against a human immunodeficiency virus (HIV) in an HIV-infected human subject undergoing antiretroviral therapy (ART) by a method comprising: (i) administering to the human subject an immunogenically effective amount of the Ad26 vaccine; (ii) administering to the human subject an immunogenically effective amount of an MVA vaccine comprising one or more Modified Vaccinia Ankara (MVA) vectors together encoding the four HIV immunogens and a pharmaceutically acceptable carrier; and (iii) administering to the human subject an effective amount of two or more anti-HIV broadly neutralizing antibodies (bNAbs) and a pharmaceutically acceptable carrier. In another aspect, the invention provides an MVA vaccine comprising one or more Modified Vaccinia Ankara (MVA) vectors together encoding four HIV immunogens having the amino acid sequences of SEQ ID NO: 1, SEQ ID NO: 2, SEQ ID NO: 3, and SEQ ID NO: 4 and a pharmaceutically acceptable carrier for use in inducing an immune response against a human immunodeficiency virus (HIV) in an HIV-infected human subject undergoing antiretroviral therapy (ART) by a method comprising: (i) administering to the human subject an immunogenically effective amount of an Ad26 vaccine comprising one or more adenovirus 26 (Ad26) vectors together encoding the four HIV immunogens, and a pharmaceutically acceptable carrier; (ii) administering to the human subject an immunogenically effective amount of the MVA vaccine; and (iii) administering to the human subject an effective amount of two or more anti-HIV broadly neutralizing antibodies (bNAbs) and a pharmaceutically acceptable carrier. In another aspect, the invention provides two or more anti-HIV broadly neutralizing antibodies (bNAbs) for use in inducing an immune response against a human immunodeficiency virus (HIV) in an HIV-infected human subject undergoing antiretroviral therapy (ART) by a method comprising: (i) administering to the human subject an immunogenically effective amount of an Ad26 vaccine comprising one or more adenovirus 26 (Ad26) vectors together encoding four HIV immunogens having the amino acid sequences of SEQ ID NO: 1, SEQ ID NO: 2, SEQ ID NO: 3, and SEQ ID NO: 4, and a pharmaceutically acceptable carrier; (ii) administering to the human subject an immunogenically effective amount of an MVA vaccine comprising one or more Modified Vaccinia Ankara (MVA) vectors together encoding the four HIV immunogens and a pharmaceutically acceptable carrier; and (iii) administering to the human subject an effective amount of the anti-HIV broadly neutralizing antibody (bNAb) and a pharmaceutically acceptable carrier.

All aspects and embodiments of the invention as described herein with respect to methods of inducing an immune response against a human immunodeficiency virus (HIV) in an HIV-infected human subject undergoing antiretroviral therapy (ART) can be applied to the vaccine and two or more anti-HIV bNAbs combinations for use and/or uses of the vaccine and two or more anti-HIV bNAbs combination in the manufacture of a medicament for inducing an immune response against HIV in an HIV-infected subject undergoing ART.

A clinical improvement of a treated HIV-infected human above a comparator HIV-infected human treated with a standard of care is expected. The clinical improvement can include one or more of a lower peak viral load, a lower chronic set point, or an increased delay in viral rebound.

In some embodiments, the method as described herein has an effect on treatment of the HIV infection, for example, as determined by a lower peak viral load as compared to standard therapies, e.g., ART only. As is commonly understood in the art, comparison of a first peak viral load in a first HIV-infected human and a second peak viral load in a second HIV-infected human is measured during the same time period. In some embodiments, the measurement is performed after cessation of all antiviral therapies. In some embodiments, the viral load is maintained at an undetectable level in a first HIV-infected human after treatment with ART, two or more anti-HIV bNAbs, and an HIV vaccine.

The following examples are to further illustrate the nature of the application. It should be understood that the following examples do not limit the application and the scope of the application is to be determined by the appended claims.

### EXAMPLES

### Example 1: Study of HIV Vaccine Regimen in Combination with Broadly Neutralizing Antibodies in HIV-Infected Humans Undergoing Antiretroviral Therapy (ART)

In Study VAC89220HTX1001 (hereafter referred to as HTX1001), the adenovirus serotype 26 (Ad26)/Modified Vaccinia Ankara (MVA) vaccines expressing mosaic HIV-1 immunogens induced robust T-cell magnitude and breadth in HIV-infected individuals on ART (Colby et al, 2020). However, although the Ad26/MVA vaccines effectively induced cellular immunity, it failed to elicit broadly neutralizing antibodies (bNAbs). Indeed, increasing evidence suggests a role of antibodies in control of the viral reservoir by specifically recruiting antibody fragment crystallizable (Fc)—dependent anti-viral activities such as antibody-dependent cellular phagocytosis (ADCP) or antibody-dependent cellular cytotoxicity (ADCC). Interestingly, ADCC-inducing antibodies are enriched in spontaneous controllers of HIV-1. Furthermore, it has been proposed that bNAbs have the potential to expand vaccine-induced T-cell responses either by slowing viral rebound and thus allowing controlled virus-induced expansion of T-cell responses, or alternatively by forming immune complexes and augmenting T-cell responses by a vaccinal effect (Niessl 2020; Nishimura 2017).

In a study in SIV-infected rhesus macaques on ART, administration of a toll-like receptor (TLR) 7 agonist in combination with the bNAb PGT121 to SIV-infected rhesus macaques on ART resulted in delayed viral rebound and post viral rebound control following ART discontinuation (Borducchi et al, 2018).

A similar administration of an HIV-1 vaccine regimen in sequential combination with bNAbs may help to achieve sustained virologic control (below 50 copies/mL) and/or reduce the size of the viral reservoir after initial viral rebound following ART interruption in humans infected with HIV-1 on ART.

The present inventors propose that combining both active and passive immunization, via use of therapeutic adenovirus and poxvirus vaccines and a combination of bNAbs, may have the ability to induce long-term ART-free virologic control.

Clinical studies in humans are conducted to investigate the effect of safety, tolerability, immunogenicity and efficacy of a vaccine regimen consisting of an adenovirus 26 (Ad26) vector prime and a boost with an MVA vector in combination with broadly neutralizing antibodies (bNAbs) PGT121, PGDM1400 and VRC07-523LS in human immunodeficiency virus type 1 (HIV-1)—infected study participants on suppressive anti-retroviral therapy (ART).

### Objectives

This study evaluates the safety and tolerability of a vaccine regimen containing Ad26.Mos4.HIV and MVA-BN-HIV and the broadly neutralizing antibodies (bNAbs) PGT121, PGDM1400 and VRC07-523LS in HIV-1-infected adults. Additionally, the study assesses the magnitude, breadth, functionality and specificity of the cellular and humoral responses elicited by a vaccine regimen containing Ad26.Mos4.HIV and MVA-BN-HIV followed by the bNAbs PGT121, PGDM1400 and VRC07-523LS.

TABLE 1
objectives and endpoints

| | |
|---|---|
| Objectives | Endpoints |

Primary

| | |
|---|---|
| To assess safety/tolerability of a vaccine | Solicited local and systemic adverse events (AEs) for 7 |
| regimen containing Ad26.Mos4.HIV and | days after each vaccination and bNAb administration |
| MVA-BN-HIV and /or the broadly | SAEs and MAAEs during the course of the study |
| neutralizing antibodies (bNAbs) PGT121, | Unsolicited AEs for 28 days after vaccination and 56 |
| PGDM1400 and VRC07-523LS in HIV-1- | days after bNAb administration |
| infected adults. | |
| To assess the magnitude, breadth, | ELISPOT response and magnitude following stimulus by |
| functionality and specificity of the cellular | peptide pools covering Env, Gag, or Pol will be evaluated |
| and humoral responses elicited by a vaccine | by standard criteria. |
| regimen containing Ad26.Mos4.HIV and | Total immunoglobulin G (IgG) and subclass (IgG1-4) |
| MVA-BN-HIV followed by the bNAbs | specific antibody titers to envelope (Env) proteins |
| PGT121, PGDM1400 and VRC07-523LS | representing Clades A, B, and C, as well as Mosaic |
| or placebo in HIV-1-infected adults. | antigens. |
| | Antibody functionality assessment by antibody- |
| | dependent cell-mediated phagocytosis (ADCP). |
| To assess sustained virologic suppression | The proportion of participants who remain off ART |
| after a vaccine regimen containing | maintaining plasma HIV RNA <1000 cps/mL for at least |
| Ad26.Mos4.HIV and MVA-BN-HIV | two thirds of the time between 24 and 72 weeks of |
| followed by the bNAbs PGT121, | analytical treatment interruption (ATI). |
| PGDM1400 and VRC07-523LS versus | |
| bNAbs alone in HIV-1-infected adults. | |

Secondary

| | |
|---|---|
| To assess the effect of a vaccine regimen | Changes in inducible HIV reservoir by Q2VO, and |
| containing Ad26.Mos4.HIV and MVA- | changes in intact proviral DNA levels by multiplex PCR- |
| BN-HIV followed by the bNAbs PGT121, | based assay (IPDA) from baseline to week 24 (start |
| PGDM1400 and VRC07-523LS versus | bNAbs) to week 36 (4 weeks following lastbNAb). |
| vaccines or bNAbs alone on the inducible | |
| HIV reservoir. | |
| To determine the effect of PGT121, | Phylogenetic and phenotypic comparison of viruses |
| VRC07-523LS and PGDM1400 on viral | grown from PBMCs collected from volunteers while |
| escape during the analytical treatment | on ART to rebound viruses collected after ART |
| interruption period. | interruption. |
| To compare the time to viral rebound | The proportion of participants in group 2 who remain |
| (defined as confirmed plasma HIV RNA | off ART maintaining plasma HIV RNA <1000 |
| levels ≥1,000 copies/mL) following | cps/mL at week 48 compared to historical controls |
| vaccine regimen containing | (i.e., in the A5345 study). |
| Ad26.Mos4.HIV, MVA-BN-HIV and | |
| placebo with the results of the rates of | |
| viral rebound as observed in historical | |
| controls (i.e., in the A5345 study; | |
| NCT03001128). | |
| To evaluate PGT121, VRC07-523LS | Serum levels of PGT121, VRC07-523LS and |
| and PGDM1400 serum levels at which | PGDM1400 at time of viral rebound |
| viral rebound is detected during the | |
| analytical treatment interruption | |

Exploratory

| | |
|---|---|
| To assess changes in HIV-1 reservoir | Single HIV RNA copy assay in samples with HIV RNA |
| between the 3 groups from baseline to | <50 copies/mL. |
| week 24 to time of viral rebound. | Cell-associated HIV DNA (total, integrated and 2 long |
| | terminal repeat circles) in total CD4⁺ T-cells, and the |
| | memory CD4⁺ subsets in PBMC and lymph node |
| | aspirates. |
| | Cell-associated HIV RNA in total CD4⁺ T-cells, and in |
| | the memory CD4⁺ subsets. |
| To assess breadth/specificity of cellular | Intracellular cytokine staining (ICS) assays with Env, |
| immune response to a vaccine regimen | group specific antigen (Gag), and/or polymerase (Pol)- |
| containing Ad26.Mos4.HIV and MVA- | peptide pools will be used to determine the magnitude, |
| BN-HIV followed by the bNAbs PGT121, | functionality and phenotype of T-cell responses elicited. |
| PGDM1400 and VRC07-523LS versus | If positive efficacy is observed, T-cell receptor analysis |
| vaccine or bNAbs alone in HIV-1-infected | might be undertaken. |
| adults | Additional ELISPOT based mapping of positive peptide |
| | pools and epitope optimal peptides to determine the |
| | number of positive epitopes for each individual. |
| | Flow cytometric analysis of lymph node aspirates will be |
| | used to determine the phenotype and specificity of T and |
| | B cellular responses. |
| To analyze the breadth and functionality of | Fine mapping of linear epitope binding antibody |
| antibodies generated in response to a | specificity as assessed by peptide binding array. |
| vaccine regimen containing | HIV neutralizing antibody (nAb) titers for Tier 1 and Tier |
| Ad26.Mos4.HIV and MVA-BN-HIV | 2 viruses covering ia, Clades A, B, and C; note: Tier 2 |
| followed by the bNAbs PGT121, | will be assessed only if Tier 1 shows positive results. |
| PGDM1400 and VRC07-523LS versus | Other antibody functionality assays may include but are |
| vaccine or bNAbs alone in HIV-1-infected | not limited to antibody-dependent cellular cytotoxicity |
| adults | (ADCC), antibody-dependent complement deposition |
| | and/or antibody-dependent cell-mediated vims |
| | inhibition. |
| | B-cell receptor analysis. |
| To analyze the durability of | Participants with detectable responses to vaccination: |
| vaccine-induced immune response to a | humoral and cellular immunogenicity assessments at |
| vaccine regimen containing | time points after the 2nd vaccination to determine the |
| Ad26.Mos4.HIV and MVA-BN-HIV | durability of responses as described for the primary |
| followed by the bNAbs PGT121, | endpoints. |
| PGDM1400 and VRC07-523LS versus | |
| vaccines alone in HIV-1-infected adults | |
| To evaluate baseline Ad26 and MVA | Ad26 nAbs (titer) at baseline. |
| serostatus in HIV-1-infected participants | Vaccinia-virus specific nAbs titer at baseline. |
| on suppressive ART. | |
| To explore the landscape of proviral | Proviral escape mutations in epitopes targeted by pre and |
| sequence variations in HIV-1-infected | post vaccine cytotoxic T lymphocytes responses. |
| adults on suppressive ART. | |
| To explore gene expression patterns | Regulation of genes (or clusters) that predict specific |
| between the different vaccine regimens in | immune responses. |
| HIV-1-infected adults on suppressive ART. | |

### Hypotheses

This study evaluates whether vaccine therapy administered in the form of 1 dose of Ad26.Mos4.HIV boosted with 1 dose of MVA-BN-HIV followed by a cocktail of broadly neutralizing antibodies (bNAbs) PGT121, PGDM1400 and VRC07-523LS 12 weeks later, versus vaccines or bNAbs alone is well-tolerated, results in a measurable immune response (vaccine+bNAbs vs. vaccine+placebo) and leads to a delay in viral rebound (vaccine+bNAbs vs. placebo+bNAbs).

### Vaccination and Experimental Design

This is a multicenter, randomized, parallel-group, placebo-controlled, double-blind, Phase 1/2a clinical study to investigate the safety, tolerability, immunogenicity and efficacy of a vaccine regimen consisting of an Ad26.Mos4.HIV prime and a boost with MVA-BN-HIV in combination with bNAbs PGT121, PGDM1400 and VRC07-523LS versus vaccines or bNAbs alone. Study vaccination and bNAbs administration will occur in addition to ART.

The study will enroll 36 adults randomized in a 1:1:1 ratio to 3 groups (vaccines+bNAbs, vaccine+placebo, placebo+bNAbs), respectively. The study population will include HIV infected adults (≥18 to ≤70 years old) who are on suppressive ART for at least 48 weeks prior to randomization. ART is defined for this study as a combination therapy regimen including at least two antiretroviral agents, e.g., two nucleoside reverse transcriptase inhibitors plus either non-nucleoside reverse transcriptase inhibitor or integrase inhibitor. Changes in ART due to tolerability concerns are allowed as long as there is documented viral load suppression data available and there cannot be any changes in ARVs for 6 weeks prior to randomization. Participants must have a plasma HIV RNA <50 cps/mL at screening and at least one documented evidence of plasma HIV RNA <50 cps/mL after the last ART change. One blip of HIV RNA ≥50 and <200 cps/mL within 12 weeks before screening is acceptable, provided that the most recent HIV RNA is <50 cps/mL. In case of ART change at screening, 1 blip of HIV RNA ≥50 and <200 cps/mL is allowed after 6 weeks on new ART regimen, provided that HIV RNA is <50 cps/mL for the repeat test. Participants must be willing to undergo analytical treatment intervention (ATI), and have the ability and willingness to restart ART according to study guidelines.

The study comprises of a screening period of 10 weeks (Stage 0), a 24-week vaccination and follow-up period (Stage 1), a 4-week bNAb administration period and a 20-week bNAb wash-out period (Stage 2), and a 24-week monitoring period (Stage 3). An ATI will be conducted during Stage 2-3. As used herein, “week X” refers to X weeks after the initial administration of the Ad26 vaccine at week 0.

### Dosage and Administration

### STAGE 1 (Week 0-24; Vaccination Dosing and Follow-Up)

After eligibility has been established, participants will move to Stage 1 and be randomized. Participants will receive intramuscular (IM) doses of study vaccine or placebo at 2 time points: adenovirus 26 vectors encoding mosaic HIV immunogens (Ad26.Mos4.HIV) or placebo will be given at Week 0; MVA vectors encoding mosaic HIV immunogens (MVA-BN-HIV) or placebo will be given at Week 12. Study vaccinations will be administered in addition to ART. Participants in both the vaccine and placebo arms will continue their suppressive ART as per current treatment guidelines until 2 days after the first dose of bNAbs.

STAGE 2 (Week 24-48; bNAb Dosing and Wash-Out; Start ART ATI)

All participants will continue their suppressive ART as per treatment guidelines until week 24 (12 weeks after vaccination 2). The participants will need to meet the individual entry criteria, including plasma HIV-1 RNA <50 copies/mL and CD4+ T-cell count ≥450 cells/mm³ within 30 days prior to Stage 2 registration and no AIDS-defining illness according to CDC criteria within the last 6 months since study randomization, to start ATI. The decision to proceed with ATI has to be made by the time of the relevant study visit including visit window.

Participants eligible to initiate ATI will receive a single intravenous (IV) dose of VRC07-523LS at 10 mg/kg and PGT121 and PGDM1400 at 20 mg/kg each, or placebo at week 24. Two days after bNAb administration, ART will be stopped. To account for the shorter plasma half-life of PGT121 and PGDM1400 compared to VRC07-523LS, participants will receive a second IV dose of PGT121 and PGDM1400 at 20 mg/kg each or placebo at week 28.

Study vaccines (Ad26.Mos4.HIV and MVA-BN-HIV), and bNAbs, and placebo with the administered doses are as follows:

(i) Ad26.Mos4.HIV is composed of the following four vaccine products supplied pre-mixed in the same vial and administered in a 1:1:1:1 ratio of viral particles (vps):Ad26.Mos1Env, Ad26.Mos2SEnv, Ad26.Mos1Gag-Pol, and Ad26.Mos2Gag-Pol encoding HIV mosaic ENV1 (SEQ ID NO: 1), mosaic Env2S (SEQ ID NO: 2), mosaic GagPol1 (SEQ ID: NO 3), and mosaic GagPol2 (SEQ ID NO: 4) genes, respectively; administered at a total dose of about 5×10¹⁰ viral particles (vp) in 0.5 mL injection;

(ii) MVA-BN-HIV is a monovalent vaccine comprising a single Modified Vaccinia Ankara vector encoding Mos1.Env (SEQ ID NO: 1), Mos2S.Env (SEQ ID NO: 2), Mos1.Gag-Pol (SEQ ID NO: 3), and Mos2.Gag-Pol (SEQ ID NO: 4); administered at a total dose about 2×10⁸ infectious units (IU) in 0.5 mL injection; MVA-BN-HIV has been described in more detail as MVA-mBN414 in example 7 of WO 2018/229711;

(iii) PGT121 is a human monoclonal antibody (mAb) that targets the HIV-1 V3 glycan, centered on N332. PGT-121 is described in U.S. Pat. No. 10,836,811, the details of which are incorporated herein by reference. It was developed by the Beth Israel Deaconess Medical Center and manufactured under cGMP standards at Catalent Pharma Solutions and is formulated at a concentration of 50 mg/mL in a buffer composed of 20 mM acetate, 9% sucrose, 0.008% polysorbate 80, at pH 5.2. Each vial contains 6 mL of PGT121 filled in a standard 10-mL glass vial. PGT121 is administered at a dose of 20 mg/kg per administration;

(iv) PGDM1400 is a human mAb that targets the HIV-1 V2 glycan, centered on N160. PGDM1400 is described in U.S. Pat. No. 10,093,720, the details of which are incorporated herein by reference. It was developed by the Beth Israel Deaconess Medical Center and manufactured under cGMP standards at Catalent Pharma Solutions and is formulated at a concentration of 50 mg/mL in buffer is composed of 20 mM Acetate, 9% sucrose, 0.008% polysorbate 80 at pH 5.2. Each vial contains 6 mL of PGDM1400 filled in a standard 10-mL glass vial. PGDM1400 is administered at a dose of 20 mg/kg per administration;

(v) VRC-HIVMAB075-00-AB (VRC07-523LS) is a human mAb that targets the HIV-1 CD4 binding site. VRC07-523LS is described in U.S. Pat. No. 9,695,230, the details of which are incorporated herein by reference. It was developed by the VRC/NIAID/NIH and manufactured under current Good Manufacturing Practice (cGMP) standards at the VRC Pilot Plant operated under contract by the Vaccine Clinical Materials Program, Leidos Biomedical Research, Inc. (Frederick, Md.). Product is provided at 100±10 mg/mL as 10 mL glass vials with a 6.25±0.1 mL fill volume and 3 mL glass vials with a 2.25 mL±0.1 mL fill volume. VRC07-523LS is administered at a dose of 10 mg/kg.

(vi) Placebo is 0.9% sodium chloride (0.5 mL injection).

In vitro data show that VRC07-523LS, PGDM1400, PGT121 are complementary in their coverage of viral strains; many of the HIV-1 strains that are resistant to one mAb are sensitive to the other(s). The combination of VRC07-523LS, PGDM1400, and PGT121 yields theoretical coverage of over 99% of HIV-1 strains on a multi-clade panel. Moreover, 86% of global viruses are sensitive to at least 2 antibodies in this cocktail, thus assuring that the majority of individuals will receive at least 2 active antibodies.

Preliminary pharmacokinetics data show that PGT121's and PGDM1400's half-life in HIV-uninfected volunteers is approximately 20 days. In HIV infected, viremic individuals the half-life for both bNAbs is between 11 and 12 days. Initial data suggests that the PK of each antibody is not affected by co-administration. VRC07-523LS half-life in HIV infected, viremic individuals is approximately 30 days.

PK simulation results suggest that the proposed study design should be able to attain desirable serum concentration levels >10 ug/ml for the initial 10 weeks after the first bNAb administration conferring sufficient neutralization efficacy based on the measured neutralization potency against diverse panels of pseudoviruses in the TZM-bl assay. In addition, this dosing regimen also guarantees sufficient wash-out of all three antibodies by week 24 following first bNAb administration to serum levels that are likely considered non-therapeutic for each bNAb based on known potency (IC50/IC80). As PGDM1400 and PGT121 showing up to 10-fold higher potency than VRC07-523LS, plasma level cut-off of <1 ug/ml for PGDM1400 and PGT121 and <10 ug/ml of VRC07-523LS were applied.

Subjects receive the study vaccines administered by intramuscular administration and bNAbs by intravenous administration or placebo according to the schedule in Table 2 below:

TABLE 2
Schedule for administration of study vaccines

| Group | N | Week 0 | Week 12 | Week 24 | Week 28 |
|---|---|---|---|---|---|
| Test | 12 | Ad26.Mos4.HIV | MVA- | PGT121 + | PGT121 + |
| Group | | | BN-HIV | PGDM1400 + | PGDM1400 |
| | | | | VRC07-523LS | |
| Control | 12 | Ad26.Mos4.HIV | MVA- | Placebo | Placebo |
| Group | | | BN-HIV | | |
| 1 | | | | | |
| Control | 12 | Placebo | Placebo | PGT121 + | PGT121 + |
| Group | | | | PGDM1400 + | PGDM1400 |
| 2 | | | | VRC07-523LS | |

Subjects in both the test and control groups continue to receive standard ART (e.g. at least two antiretroviral agents, e.g. two nucleoside reverse transcriptase inhibitors plus either non-nucleoside reverse transcriptase inhibitor or protease inhibitor or integrase inhibitor) for HIV treatment until 2 days after the administration of the first dose of bNAbs (at week 24). Blood samples will be taken at specific clinic visits to assess immune and virologic responses as well as pharmacokinetics (PK) and pharmacodynamic (PD) of bNAbs.

After the final bNAb infusion at week 28, participants will experience a bNAb washout period. This is done to allow bNAb concentrations to decay to sufficiently low levels so as not to affect viremia during stage 3 when participants are monitored for viral rebound. The expected washout period is 20 weeks based on PK modelling estimates of all 3 bNAbs.

Participants who do not qualify for ATI will continue safety and immunogenicity follow-up until the end of the main study at Week 72. Participants who qualify and start ATI will have HIV RNA monitoring biweekly until the Week 72 visit. If HIV RNA is confirmed detectable >50 cp/ml, weekly quantitative HIV RNA testing will be performed until confirmed undetectable (on 2 consecutive weekly assessments) or, until criteria to reinitiate ART are met. Assessments for safety, immunogenicity and virology will also be performed. Criteria for restart of ART will be checked at every visit.

### STAGE 3 (Week 48-72; Monitoring for Viral Rebound; Continued ART ATI)

If no ART restart criteria are met, participants will continue the ATI. If ART restart criteria are met or if the participant reaches the end of the study (week 72), ART will be initiated. Participants will be followed every 2 weeks until confirmed plasma HIV RNA levels of <50 cps/mL and then every 8 weeks until the end of the study. During these follow-up visits, safety and immunogenicity assessments will be performed, including monitoring of HIV RNA and CD4 count.

Rebound in viral replication during/post ARV ATI (HIV RNA >200 cps/mL) will not be considered an AE/SAE as it is a study primary efficacy endpoint which is captured in the clinical study database and will be analyzed as such.

After 72 weeks, the protocol will be completed, and all study participants will remain on ART. The ART regimen will be resumed prior to the end of the study (week 72) for a participant if:

1) The participant's plasma HIV-1 RNA levels are ≥1000 copies/mL for ≥4 consecutive weeks AND have not dropped 0.2 log 10 from the previous week

2) The participant's plasma HIV-1 RNA levels are ≥200 copies/mL for ≥12 consecutive weeks

3) The participant has any signs or symptoms attributable to HIV rebound other than mild fatigue (including, but not limited to, unintentional weight loss [>5% of pre-ATI body weight], otherwise unexplained persistent fever [>38° C.], persistent night sweats, persistent diarrhea, oral candidiasis, and generalized lymphadenopathy)

4) The participant has two consecutive CD4+ T cell counts <350 cells/μL at least 2 weeks apart or CD4%<15%

5) The participant has self-reported condomless anal or vaginal sex with an individual without HIV or of unknown serostatus

6) The participant has a bacterial sexually transmitted infection (STI) (i.e., syphilis, gonorrhea, chlamydia)

7) The participant develops clinical progression to Centers for Disease Control and Prevention (CDC) Stage 3 disease (https://www.cdc.gov/mmwr/preview/mmwrhtml/rr6303a1.htm?s_cid=rr6303a1_e #Appendix)

8) The participant becomes pregnant

9) The participant requests ART restart

10) ART restart is deemed medically necessary by the primary HIV care provider.

If at least one of these criteria is fulfilled, the volunteer will be asked to return prior to the next scheduled visit, and ART will be resumed. Upon confirmation of viral rebound per the criteria above, another blood sample will be collected and viral genotyping will be performed to determine if ART resistance mutations are present.

Cellular immune response assays will include, but are not limited to, interferon γ (IFNγ) ELISPOT assay, ICS, and multiparameter flow cytometry.

Humoral immune response assays will include, but are not limited to, Env-Ab-binding assays, virus neutralization assay, and assays for Ab functionality.

Additional exploratory assays to further evaluate breadth and functionality of humoral and cellular immune responses may be performed based on the outcomes of the primary analysis.

HIV RNA levels will be determined at specified time points to evaluate efficacy of the study drug interventions.

Pharmacokinetic (PK) sampling will be performed. Plasma samples will be analyzed to determine bNAb concentrations and estimate the following pharmacokinetic parameters; Elimination half-life (t1/2), Clearance (CL/F), Volume of distribution (Vz/F), Area under the concentration decay curve (AUC), Concentration at time of detectable plasma viremia as appropriate.

It is understood that the examples and embodiments described herein are for illustrative purposes only, and that changes could be made to the embodiments described above without departing from the broad inventive concept thereof. It is understood, therefore, that this application is not limited to the particular embodiments disclosed, but it is intended to cover modifications within the spirit and scope of the application as defined by the appended claims.

SEQUENCE LISTING

SEQ ID NO: 1 (Mos1.Env) 685 aa:
MRVTGIRKNYQHLWRWGTMLLGILMICSAAGKLWVTVYYGVPVWKEATTTLFCASDAKAYDTEVHNVWATHACVPTDPNPQEVVLENVTEN
FNMWKNNMVEQMHEDIISLWDQSLKPCVKLTPLCVTLNCTDDVRNVTNNATNTNSSWGEPMEKGEIKNCSFNITTSIRNKVQKQYALFYKL
DVVPIDNDSNNTNYRLISCNTSVITQACPKVSFEPIPIHYCAPAGFAILKCNDKKFNGTGPCTNVSTVQCTHGIRPVVSTQLLLNGSLAEE
EVVIRSENFTNNAKTIMVQLNVSVEINCTRPNNNTRKSIHIGPGRAFYTAGDIIGDIRQAHCNISRANWNNTLRQIV
EKLGKQFGNNKTIVFNHSSGGDPEIVMHSFNCGGEFFYCNSTKLFNSTWTWNNSTWNNTKRSNDTEEHITLPCRIKQIINMWQEVGKAMYA
PPIRGQIRCSSNITGLLLTRDGGNDTSGTEIFRPGGGDMRDNWRSELYKYKVVKIEPLGVAPTKAKRRVVQSEKSAVGIGAVFLGFLGAAG
STMGAASMTLTVQARLLLSGIVQQQNNLLRAIEAQQHLLQLTVWGIKQLQARVLAVERYLKDQQLLGIWGCSGKLICTTTVPWNASWSNKS
LDKIWNNMTWMEWEREINNYTSLIYTLIEESQNQQEKNEQELLELDKWASLWNWFDISNWLW
SEQ ID NO: 2 (Mos2SEnv) 711 aa
MRVRGMLRNWQQWWIWSSLGFWMLMIYSVMGNLWVTVYYGVPVWKDAKTTLFCASDAKAYEKEVHNVWATHACVPTDPNPQEIVLGNVTEN
FNMWKNDMVDQMHEDIISLWDASLEPCVKLTPLCVTLNCRNVRNVSSNGTYNIIHNETYKEMKNCSFNATTVVEDRKQKVHALFYRLDIVP
LDENNSSEKSSENSSEYYRLINCNTSAITQACPKVSFDPIPIHYCAPAGYAILKCNNKTFNGTGPCNNVSTVQCTHGIKPVVSTQLLLNGS
LAEEEIIIRSENLTNNAKTIIVHLNETVNITCTRPNNNTRKSIRIGPGQTFYATGDIIGDIRQAHCNLSRDGWNKTLQGVKKKLAEHFPNK
TIKFAPHSGGDLEITTHTFNCRGEFFYCNTSNLFNESNIERNDSIITLPCRIKQIINMWQEVGRAIYAPPIAGNITCRSNITGLLLTRDGG
SNNGVPNDTETFRPGGGDMRNNWRSELYKYKVVEVKPLGVAPTEAKRRVVEREKRAVGIGAVFLGILGAAGSTMGAASITLTVQARQLLSG
IVQQQSNLLRAIEAQQHMLQLTVWGIKQLQTRVLAIERYLQDQQLLGLWGCSGKLICTTAVPWNTSWSNKSQTDIWDNMTWMQWDKEIGNY
TGEIYRLLEESQNQQEKNEKDLLALDSWNNLWNWFSISKWLWYIKIFIMIVGGLIGLRIIFAVLSIVNRVRQGY
SEQ ID NO: 3 (Mos1.Gag-Pol) 1350 aa:
MGARASVLSGGELDRWEKIRLRPGGKKKYRLKHIVWASRELERFAVNPGLLETSEGCRQILGQLQPSLQTGSEELRSLYNTVATLYCVHQR
IEIKDTKEALEKIEEEQNKSKKKAQQAAADTGNSSQVSQNYPIVQNIQGQMVHQAISPRTLNAWVKVVEEKAFSPEVIPMFSALSEGATPQ
DLNTMLNTVGGHQAAMQMLKETINEEAAEWDRVHPVHAGPIAPGQMREPRGSDIAGTTSTLQEQIGWMTNNPPIPVGEIYKRWIILGLNKI
VRMYSPVSILDIRQGPKEPFRDYVDRFYKTLRAEQASQDVKNWMTETLLVQNANPDCKTILKALGPAATLEEMMTACQGVGGPGHKARVLA
EAMSQVTNSATIMMQRGNFRNQRKTVKCFNCGKEGHIAKNCRAPRKKGCWKCGKEGHQMKDCTERQANFLGKIWPSNKGRPGNFLQNRPEP
TAPPEESFRFGEETTTPSQKQEPIDKEMYPLASLKSLFGNDPSSQMAPISPIETVPVKLKPGMDGPRVKQWPLTEEKIKALTAICEEMEKE
GKITKIGPENPYNTPVFAIKKKDSTKWRKLVDFRELNKRTQDFWEVQLGIPHPAGLKKKKSVTVLAVGDAYFSVPLDEGFRKYTAFTIPST
NNETPGIRYQYNVLPQGWKGSPAIFQCSMTRILEPFRAKNPEIVIYQYMAALYVGSDLEIGQHRAKIEELREHLLKWGFTTPDKKHQKEPP
FLWMGYELHPDKWTVQPIQLPEKDSWTVNDIQKLVGKLNWASQIYPGIKVRQLCKLLRGAKALTDIVPLTEEAELELAENREILKEPVHGV
YYDPSKDLIAEIQKQGHDQWTYQIYQEPFKNLKTGKYAKMRTAHTNDVKQLTEAVQKIAMESIVIWGKTPKFRLPIQKETWETWWTDYWQA
TWIPEWEFVNTPPLVKLWYQLEKDPIAGVETFYVAGAANRETKLGKAGYVTDRGRQKIVSLTETTNQKTALQAIYLALQDSGSEVNIVTAS
QYALGIIQAQPDKSESELVNQIIEQLIKKERVYLSWVPAHKGIGGNEQVDKLVSSGIRKVLFLDGIDKAQEEHEKYHSNWRAMASDFNLPP
VVAKEIVASCDQCQLKGEAMHGQVDCSPGIWQLACTHLEGKIILVAVHVASGYIEAEVIPAETGQETAYFILKLAGRWPVKVIHTANGSNF
TSAAVKAACWWAGIQQEFGIPYNPQSQGVVASMNKELKKIIGQVRDQAEHLKTAVQMAVFIHNFKRKGGIGGYSAGERIIDIIATDIQTKE
LQKQIIKIQNFRVYYRDSRDPIWKGPAKLLWKGEGAVVIQDNSDIKVVPRRKVKIIKDYGKQMAGADCVAGRQDED
SEQ ID NO: 4 (Mos2.Gag-Pol) 1341 aa:
MGARASILRGGKLDKWEKIRLRPGGKKHYMLKHLVWASRELERFALNPGLLETSEGCKQIIKQLQPALQTGTEELRSLFNTVATLYCVHAE
IEVRDTKEALDKIEEEQNKSQQKTQQAKEADGKVSQNYPIVQNLQGQMVHQPISPRTLNAWVKVIEEKAFSPEVIPMFTALSEGATPQDLN
TMLNTVGGHQAAMQMLKDTINEEAAEWDRLHPVHAGPVAPGQMREPRGSDIAGTTSNLQEQIAWMTSNPPIPVGDIYKRWIILGLNKIVRM
YSPTSILDIKQGPKEPFRDYVDRFFKTLRAEQATQDVKNWMTDTLLVQNANPDCKTILRALGPGATLEEMMTACQGVGGPSHKARVLAEAM
SQTNSTILMQRSNFKGSKRIVKCFNCGKEGHIARNCRAPRKKGCWKCGKEGHQMKDCTERQANFLGKIWPSHKGRPGNFLQSRPEPTAPPA
ESFRFEETTPAPKQEPKDREPLTSLRSLEGSDPLSQMAPISPIETVPVKLKPGMDGPKVKQWPLTEEKIKALVEICTEMEKEGKISKIGPE
NPYNTPIFAIKKKDSTKWRKLVDFRELNKRTQDFWEVQLGIPHPAGLKKKKSVTVLAVGDAYFSVPLDEDFRKYTAFTIPSINNETPGIRY
QYNVLPQGWKGSPAIFQSSMTKILEPFRKQNPDIVIYQYMAALYVGSDLEIGQHRTKIEELRQHLLRWGFTTPDKKHQKEPPFLWMGYELH
PDKWTVQPIVLPEKDSWTVNDIQKLVGKLNWASQIYAGIKVKQLCKLLRGTKALTEVVPLTEEAELELAENREILKEPVHGVYYDPSKDLI
AEIQKQGQGQWTYQIYQEPFKNLKTGKYARMRGAHTNDVKQLTEAVQKIATESIVIWGKTPKFKLPIQKETWEAWWTEYWQATWIPEWEFV
NTPPLVKLWYQLEKEPIVGAETFYVAGAANRETKLGKAGYVTDRGRQKVVSLTDTTNQKTALQAIHLALQDSGLEVNIVTASQYALGIIQA
QPDKSESELVSQIIEQLIKKEKVYLAWVPAHKGIGGNEQVDKLVSRGIRKVLFLDGIDKAQEEHEKYHSNWRAMASEFNLPPIVAKEIVAS
CDKCQLKGEAIHGQVDCSPGIWQLACTHLEGKVILVAVHVASGYIEAEVIPAETGQETAYFLLKLAGRWPVKTIHTANGSNFTSATVKAAC
WWAGIKQEFGIPYNPQSQGVVASINKELKKIIGQVRDQAEHLKTAVQMAVFIHNFKRKGGIGEYSAGERIVDIIASDIQTKELQKQITKIQ
NFRVYYRDSRDPLWKGPAKLLWKGEGAVVIQDNSDIKVVPRRKAKIIRDYGKQMAGDDCVASRQDED
SEQ ID NO: 5 (Mos1.Env DNA)
ATGCGGGTGACCGGCATCCGGAAGAACTACCAGCACCTGTGGCGGTGGGGCACCATGCTGCTGGGCATCCTGATGATTTGCTCTGCCGCCG
GAAAGCTGTGGGTGACCGTGTACTACGGCGTGCCCGTGTGGAAAGAGGCCACCACCACCCTGTTCTGCGCCAGCGACGCCAAGGCCTACGA
CACCGAGGTGCACAACGTGTGGGCCACCCACGCCTGCGTGCCCACCGACCCCAACCCCCAGGAAGTGGTCCTGGAAAACGTGACCGAGAAC
TTCAACATGTGGAAGAACAACATGGTGGAGCAGATGCACGAGGACATCATCAGCCTGTGGGACCAGAGCCTGAAGCCCTGCGTGAAGCTGA
CCCCCCTGTGCGTGACCCTGAACTGCACCGACGACGTGCGGAACGTGACCAACAACGCCACCAACACCAACAGCAGCTGGGGCGAGCCTAT
GGAAAAGGGCGAGATCAAGAACTGCAGCTTCAACATCACCACCTCCATCCGGAACAAGGTGCAGAAGCAGTACGCCCTGTTCTACAAGCTG
GACGTGGTGCCCATCGACAACGACAGCAACAACACCAACTACCGGCTGATCAGCTGCAACACCAGCGTGATCACCCAGGCCTGCCCCAAGG
TGTCCTTCGAGCCCATCCCCATCCACTACTGCGCCCCTGCCGGCTTCGCCATCCTGAAGTGCAACGACAAGAAGTTCAACGGCACCGGCCC
CTGCACCAACGTGAGCACCGTGCAGTGCACCCACGGCATCCGGCCCGTGGTGTCCACCCAGCTGCTGCTGAACGGCAGCCTGGCCGAGGAA
GAGGTGGTGATCAGAAGCGAGAATTTCACCAACAATGCCAAGACCATCATGGTGCAGCTGAACGTGAGCGTGGAGATCAACTGCACCCGGC
CCAACAACAACACCCGGAAGAGCATCCACATCGGCCCTGGCAGGGCCTTCTACACAGCCGGCGACATCATCGGCGACATCCGGCAGGCCCA
CTGCAACATCAGCCGGGCCAACTGGAACAACACCCTGCGGCAGATCGTGGAGAAGCTGGGCAAGCAGTTCGGCAACAACAAGACCATCGTG
TTCAACCACAGCAGCGGCGGAGACCCCGAGATCGTGATGCACAGCTTCAACTGTGGCGGCGAGTTCTTCTACTGCAACAGCACCAAGCTGT
TCAACAGCACCTGGACCTGGAACAACTCCACCTGGAATAACACCAAGCGGAGCAACGACACCGAAGAGCACATCACCCTGCCCTGCCGGAT
CAAGCAGATTATCAATATGTGGCAGGAGGTCGGCAAGGCCATGTACGCCCCTCCCATCCGGGGCCAGATCCGGTGCAGCAGCAACATCACC
GGCCTGCTGCTGACCCGGGACGGCGGCAACGATACCAGCGGCACCGAGATCTTCCGGCCTGGCGGCGGAGATATGCGGGACAACTGGCGGA
GCGAGCTGTACAAGTACAAGGTGGTGAAGATCGAGCCCCTGGGCGTGGCTCCCACCAAGGCCAAGCGGCGGGTGGTGCAGAGCGAGAAGAG
CGCCGTGGGCATCGGCGCCGTGTTTCTGGGCTTCCTGGGAGCCGCCGGAAGCACCATGGGAGCCGCCAGCATGACCCTGACCGTGCAGGCC
CGGCTGCTGCTGTCCGGCATCGTGCAGCAGCAGAACAACCTGCTCCGGGCCATCGAGGCCCAGCAGCACCTGCTGCAGCTGACCGTGTGGG
GCATCAAGCAGCTGCAGGCCAGGGTGCTGGCCGTGGAGAGATACCTGAAGGATCAGCAGCTCCTGGGGATCTGGGGCTGCAGCGGCAAGCT
GATCTGCACCACCACCGTGCCCTGGAACGCCAGCTGGTCCAACAAGAGCCTGGACAAGATCTGGAACAATATGACCTGGATGGAATGGGAG
CGCGAGATCAACAATTACACCAGCCTGATCTACACCCTGATCGAGGAAAGCCAGAACCAGCAGGAAAAGAACGAGCAGGAACTGCTGGAAC
TGGACAAGTGGGCCAGCCTGTGGAACTGGTTCGACATCAGCAACTGGCTGTGG
SEQ ID NO: 6 (Mos2SEnv DNA)
ATGAGAGTGCGGGGCATGCTGAGAAACTGGCAGCAGTGGTGGATCTGGTCCAGCCTGGGCTTCTGGATGCTGATGATCTACAGCGTGATGG
GCAACCTGTGGGTCACCGTGTACTACGGCGTGCCCGTGTGGAAGGACGCCAAGACCACCCTGTTTTGCGCCTCCGATGCCAAGGCCTACGA
GAAAGAGGTGCACAACGTCTGGGCCACCCACGCCTGTGTGCCCACCGACCCCAATCCCCAGGAAATCGTCCTGGGCAACGTGACCGAGAAC
TTCAACATGTGGAAGAACGACATGGTCGATCAGATGCACGAGGACATCATCTCCCTGTGGGACGCCTCCCTGGAACCCTGCGTGAAGCTGA
CCCCTCTGTGCGTGACCCTGAACTGCCGGAACGTGCGCAACGTGTCCAGCAACGGCACCTACAACATCATCCACAACGAGACATACAAAGA
GATGAAGAACTGCAGCTTCAACGCTACCACCGTGGTCGAGGACCGGAAGCAGAAGGTGCACGCCCTGTTCTACCGGCTGGACATCGTGCCC
CTGGACGAGAACAACAGCAGCGAGAAGTCCTCCGAGAACAGCTCCGAGTACTACAGACTGATCAACTGCAACACCAGCGCCATCACCCAGG
CCTGCCCCAAGGTGTCCTTCGACCCTATCCCCATCCACTACTGCGCCCCTGCCGGCTACGCCATCCTGAAGTGCAACAACAAGACCTTCAA
TGGCACCGGCCCCTGCAACAATGTGTCCACCGTGCAGTGCACCCACGGCATCAAGCCCGTGGTGTCTACCCAGCTGCTGCTGAACGGCAGC
CTGGCCGAGGAAGAGATCATTATCAGAAGCGAGAACCTGACCAACAACGCCAAAACCATCATCGTCCACCTGAACGAAACCGTGAACATCA
CCTGTACCCGGCCTAACAACAACACCCGGAAGTCCATCCGGATCGGCCCTGGCCAGACCTTTTACGCCACCGGCGATATTATCGGCGACAT
CCGGCAGGCCCACTGCAATCTGAGCCGGGACGGCTGGAACAAGACACTGCAGGGCGTCAAGAAGAAGCTGGCCGAACACTTCCCTAACAAG
ACTATCAAGTTCGCCCCTCACTCTGGCGGCGACCTGGAAATCACCACCCACACCTTCAACTGTCGGGGCGAGTTCTTCTACTGCAATACCT
CCAACCTGTTCAACGAGAGCAACATCGAGCGGAACGACAGCATCATCACACTGCCTTGCCGGATCAAGCAGATTATCAATATGTGGCAGGA
AGTGGGCAGAGCCATCTACGCCCCTCCAATCGCCGGCAACATCACATGCCGGTCCAATATCACCGGCCTGCTGCTCACCAGAGATGGCGGC
TCCAACAATGGCGTGCCAAACGACACCGAGACATTCAGACCCGGCGGAGGCGACATGCGGAACAATTGGCGGAGCGAGCTGTACAAGTACA
AGGTGGTGGAAGTGAAGCCCCTGGGCGTGGCCCCTACCGAGGCCAAGAGAAGAGTGGTCGAACGCGAGAAGCGGGCCGTGGGAATCGGAGC
CGTGTTTCTGGGAATCCTGGGAGCCGCTGGCTCTACCATGGGCGCTGCCTCTATCACCCTGACAGTGCAGGCCAGACAGCTGCTCAGCGGC
ATCGTGCAGCAGCAGAGCAACCTGCTGAGAGCCATTGAGGCCCAGCAGCACATGCTGCAGCTGACCGTGTGGGGCATTAAGCAGCTCCAGA
CACGGGTGCTGGCCATCGAGAGATACCTGCAGGATCAGCAGCTCCTGGGCCTGTGGGGCTGTAGCGGCAAGCTGATCTGTACCACCGCCGT
GCCCTGGAATACCTCTTGGAGCAACAAGAGCCAGACCGACATCTGGGACAACATGACCTGGATGCAGTGGGACAAAGAAATCGGCAACTAT
ACCGGCGAGATCTATAGACTGCTGGAAGAGTCCCAGAACCAGCAGGAAAAGAACGAGAAGGACCTGCTGGCCCTGGATTCTTGGAACAATC
TGTGGAACTGGTTCAGCATCTCCAAGTGGCTGTGGTACATCAAGATCTTCATCATGATCGTGGGCGGCCTGATCGGCCTGCGGATCATCTT
TGCCGTGCTGAGCATCGTGAACCGCGTGCGGCAGGGCTAC
SEQ ID NO: 7 (Mos1.Gag-Pol DNA)
ATGGGAGCCAGAGCCAGCGTGCTGTCCGGAGGGGAGCTGGACCGCTGGGAGAAGATCAGGCTGAGGCCTGGAGGGAAGAAGAAGTACAGGC
TGAAGCACATCGTGTGGGCCAGCAGAGAGCTGGAACGGTTTGCCGTGAACCCTGGCCTGCTGGAAACCAGCGAGGGCTGTAGGCAGATTCT
GGGACAGCTGCAGCCCAGCCTGCAGACAGGCAGCGAGGAACTGCGGAGCCTGTACAACACCGTGGCCACCCTGTACTGCGTGCACCAGCGG
ATCGAGATCAAGGACACCAAAGAAGCCCTGGAAAAGATCGAGGAAGAGCAGAACAAGAGCAAGAAGAAAGCCCAGCAGGCTGCCGCTGACA
CAGGCAACAGCAGCCAGGTGTCCCAGAACTACCCCATCGTGCAGAACATCCAGGGACAGATGGTGCACCAGGCCATCAGCCCTCGGACCCT
GAACGCCTGGGTGAAGGTGGTGGAGGAAAAGGCCTTCAGCCCTGAGGTGATCCCCATGTTCTCTGCCCTGAGCGAGGGAGCCACACCCCAG
GACCTGAACACCATGCTGAACACCGTGGGAGGGCACCAGGCTGCCATGCAGATGCTGAAAGAGACAATCAACGAGGAAGCTGCCGAGTGGG
ACAGGGTCCACCCAGTGCACGCTGGACCTATCGCTCCTGGCCAGATGAGAGAGCCCAGAGGCAGCGATATTGCTGGCACCACCTCCACACT
GCAGGAACAGATCGGCTGGATGACCAACAACCCTCCCATCCCTGTGGGAGAGATCTACAAGCGGTGGATCATTCTGGGACTGAACAAGATC
GTGCGGATGTACAGCCCTGTGAGCATCCTGGACATCAGGCAGGGACCCAAAGAGCCCTTCAGGGACTACGTGGACCGGTTCTACAAGACCC
TGAGAGCCGAGCAGGCCAGCCAGGACGTGAAGAACTGGATGACCGAGACACTGCTGGTGCAGAACGCCAACCCTGACTGCAAGACCATCCT
GAAAGCCCTGGGACCTGCTGCCACCCTGGAAGAGATGATGACAGCCTGCCAGGGAGTGGGAGGACCTGGCCACAAGGCCAGGGTGCTGGCC
GAGGCCATGAGCCAGGTGACCAACTCTGCCACCATCATGATGCAGAGAGGCAACTTCCGGAACCAGAGAAAGACCGTGAAGTGCTTCAACT
GTGGCAAAGAGGGACACATTGCCAAGAACTGCAGGGCTCCCAGGAAGAAAGGCTGCTGGAAGTGCGGAAAAGAAGGCCACCAGATGAAGGA
CTGCACCGAGAGGCAGGCCAACTTCCTGGGCAAGATCTGGCCTAGCAACAAGGGCAGGCCTGGCAACTTCCTGCAGAACAGACCCGAGCCC
ACCGCTCCTCCCGAGGAAAGCTTCCGGTTTGGCGAGGAAACCACCACCCCTAGCCAGAAGCAGGAACCCATCGACAAAGAGATGTACCCTC
TGGCCAGCCTGAAGAGCCTGTTCGGCAACGACCCCAGCAGCCAGATGGCTCCCATCAGCCCAATCGAGACAGTGCCTGTGAAGCTGAAGCC
TGGCATGGACGGACCCAGGGTGAAGCAGTGGCCTCTGACCGAGGAAAAGATCAAAGCCCTGACAGCCATCTGCGAGGAAATGGAAAAAGAG
GGCAAGATCACCAAGATCGGACCCGAGAACCCCTACAACACCCCTGTGTTCGCCATCAAGAAGAAAGACAGCACCAAGTGGAGGAAACTGG
TGGACTTCAGAGAGCTGAACAAGCGGACCCAGGACTTCTGGGAGGTGCAGCTGGGCATCCCTCACCCTGCTGGCCTGAAGAAAAAGAAAAG
CGTGACCGTGCTGGCTGTGGGAGATGCCTACTTCAGCGTGCCTCTGGACGAGGGCTTCCGGAAGTACACAGCCTTCACCATCCCCAGCACC
AACAACGAGACACCTGGCATCAGATACCAGTACAACGTGCTGCCTCAGGGCTGGAAAGGCAGCCCTGCCATCTTCCAGTGCAGCATGACCA
GAATCCTGGAACCCTTCAGAGCCAAGAACCCTGAGATCGTGATCTACCAGTATATGGCTGCCCTCTACGTGGGCAGCGACCTGGAAATCGG
ACAGCACAGAGCCAAAATCGAAGAACTCCGCGAGCACCTGCTGAAGTGGGGATTCACCACCCCTGACAAGAAGCACCAGAAAGAGCCTCCC
TTCCTGTGGATGGGCTACGAGCTGCACCCTGACAAGTGGACCGTGCAGCCCATCCAGCTGCCAGAGAAGGACTCCTGGACCGTGAACGACA
TCCAGAAACTGGTCGGCAAGCTGAACTGGGCCAGCCAGATCTACCCTGGCATCAAAGTCAGACAGCTGTGTAAGCTGCTGAGGGGAGCCAA
AGCACTGACCGACATCGTGCCTCTGACAGAAGAAGCCGAGCTGGAACTGGCCGAGAACAGAGAGATCCTGAAAGAACCCGTGCACGGAGTG
TACTACGACCCCTCCAAGGACCTGATTGCCGAGATCCAGAAACAGGGACACGACCAGTGGACCTACCAGATCTATCAGGAACCTTTCAAGA
ACCTGAAAACAGGCAAGTACGCCAAGATGCGGACAGCCCACACCAACGACGTGAAGCAGCTGACCGAAGCCGTGCAGAAAATCGCCATGGA
AAGCATCGTGATCTGGGGAAAGACACCCAAGTTCAGGCTGCCCATCCAGAAAGAGACATGGGAAACCTGGTGGACCGACTACTGGCAGGCC
ACCTGGATTCCCGAGTGGGAGTTCGTGAACACCCCACCCCTGGTGAAGCTGTGGTATCAGCTGGAAAAGGACCCTATCGCTGGCGTGGAGA
CATTCTACGTGGCTGGAGCTGCCAACAGAGAGACAAAGCTGGGCAAGGCTGGCTACGTGACCGACAGAGGCAGACAGAAAATCGTGAGCCT
GACCGAAACCACCAACCAGAAAACAGCCCTGCAGGCCATCTATCTGGCACTGCAGGACAGCGGAAGCGAGGTGAACATCGTGACAGCCAGC
CAGTATGCCCTGGGCATCATCCAGGCCCAGCCTGACAAGAGCGAGAGCGAGCTGGTGAACCAGATCATCGAGCAGCTGATCAAGAAAGAAC
GGGTGTACCTGAGCTGGGTGCCAGCCCACAAGGGCATCGGAGGGAACGAGCAGGTGGACAAGCTGGTGTCCAGCGGAATCCGGAAGGTGCT
GTTCCTGGACGGCATCGATAAAGCCCAGGAAGAGCACGAGAAGTACCACAGCAATTGGAGAGCCATGGCCAGCGACTTCAACCTGCCTCCC
GTGGTGGCCAAAGAAATCGTGGCCAGCTGCGACCAGTGCCAGCTGAAAGGCGAGGCCATGCACGGACAGGTGGACTGCTCCCCTGGCATCT
GGCAGCTGGCATGCACCCACCTGGAAGGCAAGATCATTCTGGTGGCCGTGCACGTGGCCAGCGGATACATCGAAGCCGAAGTGATCCCTGC
CGAGACAGGGCAGGAAACAGCCTACTTCATCCTGAAGCTGGCTGGCAGATGGCCTGTGAAGGTGATCCACACAGCCAACGGCAGCAACTTC
ACCTCTGCTGCCGTGAAGGCTGCCTGTTGGTGGGCTGGCATTCAGCAGGAATTTGGCATCCCCTACAATCCCCAGTCTCAGGGAGTGGTGG
CCAGCATGAACAAAGAGCTGAAGAAGATCATCGGACAGGTCAGGGATCAGGCCGAGCACCTGAAAACTGCCGTCCAGATGGCCGTGTTCAT
CCACAACTTCAAGCGGAAGGGAGGGATCGGAGGGTACTCTGCTGGCGAGCGGATCATCGACATCATTGCCACCGATATCCAGACCAAAGAG
CTGCAGAAACAGATCATCAAGATCCAGAACTTCAGGGTGTACTACAGGGACAGCAGGGACCCCATCTGGAAGGGACCTGCCAAGCTGCTGT
GGAAAGGCGAAGGAGCCGTCGTCATCCAGGACAACAGCGACATCAAGGTGGTGCCCAGACGGAAGGTGAAAATCATCAAGGACTACGGCAA
ACAGATGGCTGGAGCCGACTGTGTCGCTGGCAGGCAGGACGAGGAC
SEQ ID NO: 8 (Mos2.Gag-Pol DNA)
ATGGGAGCCAGAGCCAGCATCCTGCGAGGAGGGAAGCTGGACAAGTGGGAGAAGATCAGGCTGAGGCCTGGAGGGAAGAAACACTACATGC
TGAAGCACCTGGTCTGGGCCAGCAGAGAGCTGGAACGGTTTGCCCTCAATCCTGGCCTGCTGGAAACCAGCGAGGGCTGCAAGCAGATCAT
CAAGCAGCTGCAGCCTGCCCTGCAGACAGGCACCGAGGAACTGCGGAGCCTGTTCAACACCGTGGCCACCCTGTACTGCGTGCATGCCGAG
ATCGAAGTGAGGGAGACCAAAGAAGCCCTGGACAAGATCGAGGAAGAGCAGAACAAGAGCCAGCAGAAAACCCAGCAGGCCAAAGAAGCCG
ACGGCAAGGTCTCCCAGAACTACCCCATCGTGCAGAACCTGCAGGGACAGATGGTGCACCAGCCCATCAGCCCTCGGACACTGAATGCCTG
GGTGAAGGTGATCGAGGAAAAGGCCTTCAGCCCTGAGGTGATCCCCATGTTCACAGCCCTGAGCGAGGGAGCCACACCCCAGGACCTGAAC
ACCATGCTGAACACCGTGGGAGGGCACCAGGCTGCCATGCAGATGCTGAAGGACACCATCAACGAGGAAGCTGCCGAGTGGGACAGGCTGC
ACCCTGTGCACGCTGGACCTGTGGCTCCTGGCCAGATGAGAGAGCCCAGAGGCAGCGATATTGCTGGCACCACCTCCAATCTGCAGGAACA
GATCGCCTGGATGACCAGCAACCCTCCCATCCCTGTGGGAGACATCTACAAGCGGTGGATCATCCTGGGACTGAACAAGATCGTGCGGATG
TACAGCCCTACCTCCATCCTGGACATCAAGCAGGGACCCAAAGAGCCTTTCAGGGACTACGTGGACCGGTTCTTCAAGACCCTGAGAGCCG
AGCAGGCCACCCAGGACGTGAAGAACTGGATGACCGACACCCTGCTGGTGCAGAACGCCAACCCTGACTGCAAGACCATCCTGAGAGCCCT
GGGACCTGGAGCCACCCTGGAAGAGATGATGACAGCCTGCCAGGGAGTGGGAGGACCCTCTCACAAGGCTAGGGTGCTGGCCGAGGCCATG
AGCCAGACCAACAGCACCATCCTGATGCAGCGGAGCAACTTCAAGGGCAGCAAGCGGATCGTGAAGTGCTTCAACTGTGGCAAAGAGGGAC
ACATTGCCAGAAACTGTAGGGCACCCAGGAAGAAAGGCTGCTGGAAGTGCGGAAAAGAAGGCCACCAGATGAAGGACTGCACCGAGAGGCA
GGCCAACTTCCTGGGCAAGATCTGGCCTAGCCACAAGGGCAGACCTGGCAACTTCCTGCAGAGCAGACCCGAGCCCACCGCTCCTCCAGCC
GAGAGCTTCCGGTTCGAGGAAACCACCCCTGCTCCCAAGCAGGAACCTAAGGACAGAGAGCCTCTGACCAGCCTGAGAAGCCTGTTCGGCA
GCGACCCTCTGAGCCAGATGGCTCCCATCTCCCCTATCGAGACAGTGCCTGTGAAGCTGAAGCCTGGCATGGACGGACCCAAGGTGAAACA
GTGGCCTCTGACCGAGGAAAAGATCAAAGCCCTGGTGGAGATCTGTACCGAGATGGAAAAAGAGGGCAAGATCAGCAAGATCGGACCCGAG
AACCCCTACAACACCCCTATCTTCGCCATCAAGAAGAAAGACAGCACCAAGTGGAGGAAACTGGTGGACTTCAGAGAGCTGAACAAGCGGA
CCCAGGACTTCTGGGAGGTGCAGCTGGGCATCCCTCACCCTGCTGGCCTGAAGAAAAAGAAAAGCGTGACCGTGCTGGCCGTGGGAGATGC
CTACTTCAGCGTGCCTCTGGACGAGGACTTCAGAAAGTACACAGCCTTCACCATCCCCAGCATCAACAACGAGACACCTGGCATCAGATAC
CAGTACAACGTGCTGCCTCAGGGATGGAAGGGCTCTCCTGCAATCTTCCAGAGCAGCATGACCAAGATCCTGGAACCCTTCCGGAAGCAGA
ACCCTGACATCGTGATCTACCAGTACATGGCAGCCCTGTACGTCGGCAGCGACCTGGAAATCGGACAGCACCGGACCAAGATCGAAGAACT
CAGGCAGCACCTGCTGCGGTGGGGATTCACCACCCCTGACAAGAAGCACCAGAAAGAGCCTCCCTTCCTGTGGATGGGCTACGAGCTGCAC
CCAGACAAGTGGACCGTGCAGCCCATCGTGCTGCCTGAGAAGGACTCCTGGACCGTGAACGACATCCAGAAACTGGTCGGCAAGCTGAACT
GGGCCAGCCAGATCTACGCTGGCATCAAAGTGAAGCAGCTGTGTAAGCTCCTGAGAGGCACCAAAGCCCTGACCGAGGTGGTGCCACTGAC
AGAGGAAGCCGAGCTGGAACTGGCCGAGAACAGAGAGATCCTGAAAGAACCCGTGCACGGAGTGTACTACGACCCCAGCAAGGACCTGATT
GCCGAGATCCAGAAGCAGGGACAGGGACAGTGGACCTACCAGATCTACCAGGAACCCTTCAAGAACCTGAAAACAGGCAAGTACGCCAGGA
TGAGGGGAGCCCACACCAACGACGTCAAACAGCTGACCGAAGCCGTGCAGAAGATCGCCACCGAGAGCATCGTGATTTGGGGAAAGACACC
CAAGTTCAAGCTGCCCATCCAGAAAGAGACATGGGAGGCCTGGTGGACCGAGTACTGGCAGGCCACCTGGATTCCCGAGTGGGAGTTCGTG
AACACCCCACCCCTGGTGAAGCTGTGGTATCAGCTGGAAAAAGAACCCATCGTGGGAGCCGAGACATTCTACGTGGCTGGAGCTGCCAACA
GAGAGACAAAGCTGGGCAAGGCTGGCTACGTGACCGACAGAGGCAGGCAGAAAGTGGTGTCCCTGACCGATACCACCAACCAGAAAACAGC
CCTGCAGGCCATCCACCTGGCTCTGCAGGACTCTGGCCTGGAAGTGAACATCGTGACAGCCAGCCAGTATGCCCTGGGCATCATTCAGGCA
CAGCCTGACAAGAGCGAGAGCGAGCTGGTGTCTCAGATCATTGAGCAGCTGATCAAGAAAGAAAAGGTGTACCTGGCCTGGGTGCCAGCCC
ACAAGGGGATCGGAGGGAACGAGCAGGTGGACAAGCTGGTGTCCAGGGGCATCCGGAAGGTGCTGTTTCTGGACGGCATCGACAAAGCCCA
GGAAGAGCACGAGAAGTACCACAGCAATTGGAGAGCCATGGCCAGCGAGTTCAACCTGCCTCCCATCGTGGCCAAAGAAATCGTGGCCTCT
TGCGACAAGTGCCAGCTGAAAGGCGAGGCCATTCACGGACAGGTGGACTGCAGCCCAGGCATCTGGCAGCTGGCCTGCACCCACCTGGAAG
GCAAGGTGATCCTGGTGGCCGTGCACGTGGCCTCTGGATACATCGAAGCCGAAGTGATCCCTGCCGAGACAGGCCAGGAAACAGCCTACTT
CCTGCTGAAGCTGGCTGGCAGGTGGCCTGTGAAAACCATCCACACAGCCAACGGCAGCAACTTCACCTCTGCCACCGTGAAGGCTGCCTGT
TGGTGGGCTGGCATTAAGCAGGAATTTGGCATCCCCTACAACCCTCAGTCTCAGGGAGTGGTGGCCTCCATCAACAAAGAGCTGAAGAAGA
TCATCGGACAGGTCAGGGATCAGGCCGAGCATCTGAAAACAGCCGTCCAGATGGCCGTGTTCATCCACAACTTCAAGCGGAAGGGAGGGAT
CGGAGAGTACTCTGCTGGCGAGAGGATCGTGGACATTATCGCCAGCGATATCCAGACCAAAGAACTGCAGAAGCAGATCACAAAGATCCAG
AACTTCAGGGTGTACTACAGGGACAGCAGAGATCCCCTGTGGAAGGGACCTGCCAAGCTGCTGTGGAAAGGCGAAGGAGCCGTCGTCATCC
AGGACAACAGCGACATCAAGGTGGTGCCCAGACGGAAGGCCAAGATCATCAGAGACTACGGCAAACAGATGGCTGGCGACGACTGCGTCGC
CTCTAGGCAGGACGAGGAC
SEQ ID NO: 9 (PGT-121 VH)
QMQLQESGPGLVKPSETLSLTCSVSGASISDSYWSWIRRSPGKGLEWIGYVHKSGDTNYSPSLKSRVNLSLDTSKNQVSLSLVAATAADSG
KYYCARTLHGRRIYGIVAFNEWFTYFYMDVWGNGTQVTVSS
SEQ ID NO: 10 (PGT-121 VL)
SDISVAPGETARISCGEKSLGSRAVQWYQHRAGQAPSLIIYNNQDRPSGIPERFSGSPDSPFGTTATLTITSVEAGDEADYYCHIWDSRVP
TKWVFGGGTTLTVL
SEQ ID NO: 11 (PGT-121 HC)
QMQLQESGPGLVKPSETLSLTCSVSGASISDSYWSWIRRSPGKGLEWIGYVHKSGDTNYSPSLKSRVNLSLDTSKNQVSLSLVAATAADSG
KYYCARTLHGRRIYGIVAFNEWFTYFYMDVWGNGTQVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTVDKSRWQQGNVFSCSVMHEALHNHYTQKS
LSLSPGK
SEQ ID NO: 12 (PGT-121 LC)
SDISVAPGETARISCGEKSLGSRAVQWYQHRAGQAPSLIIYNNQDRPSGIPERFSGSPDSPFGTTATLTITSVEAGDEADYYCHIWDSRVP
TKWVFGGGTTLTVLGQPKAAPSVTLFPPSSEELQANKATLVCLISDFYPGAVTVAWKADSSPVKAGVETTTPSKQSNNKYAASSYLSLTPE
QWKSHKSYSCQVTHEGSTVEKTVAPTECS
SEQ ID NO: 13 (PGDM1400 VH)
QVHLTQSGPEVRKPGTSVKVSCKAPGNTLKTYDLHWVRSVPGQGLQWMGWISHEGDKKVIVERFKAKVTIDWDRSTNTAYLQLSGLTSGDT
AVYYCAKGSKHRLRDYALYDDDGALNWAVDVDYLSNLEFWGQGTAVTVSS
SEQ ID NO: 14 (PGDM1400 VL)
DFVLTQSPHSLSVTPGESASISCKSSHSLIHGDRNNYLAWYVQKPGRSPQLLIYLASSRASGVPDRFSGSGSDKDFTLKISRVETEDVGTY
YCMQGRESPWTFGQGTKVDIK
SEQ ID NO: 15 (PGDM1400 HC)
QVHLTQSGPEVRKPGTSVKVSCKAPGNTLKTYDLHWVRSVPGQGLQWMGWISHEGDKKVIVERFKAKVTIDWDRSTNTAYLQLSGLTSGDT
AVYYCAKGSKHRLRDYALYDDDGALNWAVDVDYLSNLEFWGQGTAVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQGNVFSCSVMHEA
LHNHYTQKSLSLSPGK
SEQ ID NO: 16 (PGDM1400 LC)
DFVLTQSPHSLSVTPGESASISCKSSHSLIHGDRNNYLAWYVQKPGRSPQLLIYLASSRASGVPDRFSGSGSDKDFTLKISRVETEDVGTY
YCMQGRESPWTFGQGTKVDIKRTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 17 (VRC07-523LS VH)
QVRLSQSGGQMKKPGDSMRISCRASGYEFINCPINWIRLAPGKRPEWMGWMKPRHGAVSYARQLQGRVTMTRDMYSETAFLELRSLTSDDT
AVYFCTRGKYCTARDYYNWDFEHWGQGTPVTVSS
SEQ ID NO: 18 (VRC07-523LS VL)
SLTQSPGTLSLSPGETAIISCRTSQYGSLAWYQQRPGQAPRLVIYSGSTRAAGIPDRFSGSRWGPDYNLTISNLESGDFGVYYCQQYEFFG
QGTKVQVDIK
SEQ ID NO: 19 (VRC07-523LS HC)
QVRLSQSGGQMKKPGDSMRISCRASGYEFINCPINWIRLAPGKRPEWMGWMKPRHGAVSYARQLQGRVTMTRDMYSETAFLELRSLTSDDT
AVYFCTRGKYCTARDYYNWDFEHWGQGTPVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVFSCSVLHEALHSHYTQKSLSLSPGK
SEQ ID NO: 20 (VRC07-523LS LC)
SLTQSPGTLSLSPGETAIISCRTSQYGSLAWYQQRPGQAPRLVIYSGSTRAAGIPDRFSGSRWGPDYNLTISNLESGDFGVYYCQQYEFFG
QGTKVQVDIKRTVAAPSVFIFPPSDEQLKSGTASVVCLLNNFYPREAKVQWKVDNALQSGNSQESVTEQDSKDSTYSLSSTLTLSKADYEK
HKVYACEVTHQGLSSPVTKSFNRGEC

### REFERENCES

1. US20120076812
2. Barouch et al., Nat Med 2010, 16:319-323
3. Barouch et al., Cell 155:1-9, 2013
4. WO 2017/102929
5. Fischer et al, Nat Med, 2007. 13(1): p. 100-6
6. U.S. Pat. No. 6,083,716
7. WO 2005/071093
8. WO 2010/086189
9. WO 2010085984
10. WO 2012/172277
11. WO 2018/215766
12. WO 2019/086466
13. WO 2019/086456
14. Havenga, et al., 2006, J Gen Virol 87: 2135-43
15. WO 03/104467
16. WO 2007/104792
17. Abbink et al., (2007) Virol 81(9): 4654-63
18. Mayr et al. (1975) Infection 3, 6-14
19. Mayr, A. & Danner, K. (1978), Dev. Biol. Stand. 41: 225-234
20. Mayr et al. (1978), Zentralbl. Bacteriol. (B) 167:375-390
21. WO 02/42480 (US 2003/0206926)
22. WO 03/048184 (US 2006/0159699)
23. WO 2002/24224
24. US20110159036
25. U.S. Pat. No. 8,197,825
26. WO 2011/092029
27. WO 2018/229711
28. Wagh K, et al. (2018) PLoS Pathog 14(3): e1006860
29. Huang Y, et al., Cell. 2016; 165(7):1621-31
30. Bournazos S, et al., Cell. 2016; 165(7):1609-20
31. WO 2012/158948
32. WO 2015/048770
33. WO 2012/030904
34. WO 2013/055908
35. WO 2013/086533
36. WO 2017/106346
37. WO 2019/226829
38. US 2012/0288502
39. US 2015/0361160
40. Walker et al. Nature. 477: 466-470, 2011
41. Mouquet et al. Proc. Natl. Acad. Sci. 109(47): E3268-E3277, 2012
42. Julien et al., PLoS Pathog. 9: e1003342, 2013
43. Kong et al., Nat. Struc. Mol. Biol. 20: 796-803, 2013
44. Scheid et al., Nature. 458: 636-640, 2009
45. Roben et al., J Virol. 68: 4821-4828, 1994
46. U.S. Ser. No. 10/836,811
47. Sok D, et al. Proc Natl Acad Sci USA. 2014; 111(49):17624-9
48. U.S. Ser. No. 10/093,720
49. Rudicell R S, et al. J Virol. 2014; 88(21):12669-82
50. U.S. Pat. No. 9,695,230
51. WO2020/106713
52. US 2021/0079070
53. Borducchi et al. (2018) Nature 2018; 563(7731):360-364.
54. Niessl et al (2020) Nat Med. 2020; 26(2):222-22
55. Nishimura et al (2017) Nature. 2017; 543(7646):559-563.
56. Colby et al (2020) Nat Med. 2020; 26(4):498-501.

1. A method of inducing an immune response against a human immunodeficiency virus (HIV) in an HIV-infected human subject undergoing antiretroviral therapy (ART), the method comprising:
(i) administering to the human subject an adenovirus vaccine comprising one or more adenovirus vectors together encoding HIV gag, pol, and env immunogens, and a pharmaceutically acceptable carrier;
(ii) administering to the human subject a poxvirus vaccine comprising one or more poxvirus vectors together encoding the HIV gag, pol, and env immunogens and a pharmaceutically acceptable carrier; and
(iii) administering to the human subject two or more anti-HIV broadly neutralizing antibodies (bNAbs) and a pharmaceutically acceptable carrier.

2. The method of claim 1, wherein the one or more adenovirus vectors are adenovirus 26 (Ad26) vectors.

3. The method of claim 2, wherein the adenovirus vaccine comprises a first Ad26 vector encoding the HIV immunogen of SEQ ID NO: 1, a second Ad26 vector encoding the HIV immunogen of SEQ ID NO: 2, a third Ad26 vector encoding the HIV immunogen of SEQ ID NO: 3, and a fourth Ad26 vector encoding the HIV immunogen of SEQ ID NO: 4.

4. The method of claim 1, wherein the one or more poxvirus vectors are Modified Vaccinia Ankara (MVA) vectors.

5. The method of claim 4, wherein the poxvirus vaccine consists of a single MVA vector encoding the four HIV immunogens.

6. The method of claim 1, wherein each individually of the two or more anti-HIV bNAbs binds to an epitope or region of HIV gp120 envelope selected from the group consisting of (i) CD4-binding site (CD4bs) (ii) third variable loop (V3) and/or high mannose patch comprising a N332 oligomannose glycan; (iii) second variable loop (V2) and/or Env trimer apex; (iv) gp120/gp41 interface; or (v) silent face of gp120.

7. The method of claim 6, wherein the two or more anti-HIV bNAbs are selected from the group consisting of RC01, 3BNC117, VRC01-LS, VRC07-523LS, 10-1074, PGT121, and PGDM1400.

8. The method of claim 1, wherein the one or more adenovirus vectors together are administered at a total dose of about 5×10⁹ to about 1×10¹¹ viral particles (vp) of the one or more adenovirus vectors, per administration.

9. The method of claim 1, wherein the one or more poxvirus vectors together are administered at a total dose of about 1×10⁷ to about 5×10⁸ infectious units (IU) of the one or more poxvirus vectors, per administration.

10. The method of claim 1, wherein the poxvirus vaccine is administered 8-14 weeks after the adenovirus vaccine is initially administered.

11. The method of claim 1, wherein each anti-HIV bNAb is administered at a dose of about 5-40 mg/kg of the anti-HIV bNAb, per administration.

12. The method of claim 1, wherein the two or more anti-HIV bNAbs are administered 20-30 weeks after the adenovirus vaccine is initially administered.

13. The method of claim 1, wherein the two or more anti-HIV bNAbs are administered one or two times at about 20-30 weeks after the adenovirus vaccine is initially administered.

14. A method of inducing an immune response against a human immunodeficiency virus (HIV) in an HIV-infected human subject undergoing antiretroviral therapy (ART), the method comprising:
(i) intramuscularly administering to the human subject an adenovirus 26 (Ad26) vaccine comprising one or more Ad26 vectors together encoding four HIV immunogens having the amino acid sequences of SEQ ID NO: 1, SEQ ID NO: 2, SEQ ID NO: 3, and SEQ ID NO: 4, and a pharmaceutically acceptable carrier, in a total dose of about 5×10⁹ to about 1×10¹¹ viral particles (vp) of the Ad26 vectors;
(ii) intramuscularly administering to the human subject an Modified Vaccinia Ankara (MVA) vaccine comprising one or more MVA vectors, encoding the four HIV immunogens and a pharmaceutically acceptable carrier, in a total dose of about 1×10⁷ to about 5×10⁸ infectious units (IU), of the one or more MVA vectors, wherein the MVA vaccine, is administered 8-14 weeks after the Ad26 vaccine is initially administered in step (i); and
(iii) intravenously administering to the human subject PGT121, PGDM1400, and VRC07-523LS anti-HIV broadly neutralizing antibodies (bNAbs) and one or more pharmaceutically acceptable carriers, at a dose of about 5 mg/kg to about 40 mg/kg of each anti-HIV bNAb, per administration, wherein the anti-HIV bNAbs are administered one or two times at 20-30 weeks after the Ad26 vaccine is initially administered in step (i).

15. The method of claim 14, wherein PGT121 is administered at a dose of 20 mg/kg of PGT121, per administration, at 24 and 28 weeks after the Ad26 vaccine is initially administered in step (i).

16. The method of claim 14, wherein PGDM1400 is administered at a dose of 20 mg/kg of PGDM1400, per administration, at 24 and 28 weeks after the Ad26 vaccine is initially administered in step (i).

17. The method of claim 14, wherein VRC07-523LS is administered at a dose of 10 mg/kg of VRC07-523LS at 24 weeks after the Ad26 vaccine is initially administered in step (i).

18. The method of claim 1, wherein the human subject has undergone ART for at least 48 weeks prior to being initially administered the adenovirus vaccine.

19. The method of claim 1, wherein the human subject continues undergoing suppressive ART during the treatment.

20. The method of claim 19, wherein the suppressive ART is stopped after the initial administration of two or more anti-HIV bNAbs.

21. A method of treating a human immunodeficiency virus (HIV) infection in a human subject in need thereof, comprising:
(i) treating the human subject with an antiretroviral therapy (ART); and
(ii) inducing an immune response against the HIV in the human subject using a method of claim 1.

22. The method of claim 21, further comprising: discontinuing the ART treatment of step (i) after step (ii).
